(19) United States
(12) Patent Application Publication
(10) Pub. No.: US 2018/0172689 A1
(43) Pub. Date: Jun. 21, 2018

(54) METHODS FOR DIAGNOSIS OF BLADDER CANCER

(71) Applicant: The Board of Trustees of the Leland Stanford Junior University, Stanford, CA (US)

(72) Inventors: Joseph C. Liao, Redwood City, CA (US); Kathleen E. Mach, Palo Alto, CA (US); Lai Yi Sin, Cupertino, CA (US)

(21) Appl. No.: 15/844,828

(22) Filed: Dec. 18, 2017

Related U.S. Application Data
(60) Provisional application No. 62/435,803, filed on Dec. 18, 2016.

(51) Int. Cl. G01N 33/574; G01N 33/493; A61K 39/04; C07K 14/49; A61P 35/00

(52) U.S. Cl. CPC G01N 33/57407; G01N 33/493; A61P 35/00; C07K 14/49; A61K 39/04

(57) ABSTRACT

Methods for diagnosis of bladder cancer are disclosed. In particular, the invention relates to the use of urinary biomarkers for aiding diagnosis, prognosis, and treatment of bladder cancer, and to a panel of biomarkers that can be used to distinguish high-grade bladder cancer from low-grade bladder cancer.

### CROSS-REFERENCE TO RELATED APPLICATION

This application claims benefit under 35 U.S.C. § 119(e) of provisional application Ser. No. 62/435,803, filed Dec. 18, 2016, which is hereby incorporated by reference in its entirety.

### TECHNICAL FIELD

The present invention pertains generally to methods for diagnosis of bladder cancer. In particular, the invention relates to the use of biomarkers for aiding diagnosis, prognosis, and treatment of bladder, and more specifically to biomarkers that can be used to detect high-grade as well as low-grade bladder cancer.

### BACKGROUND

Bladder cancer is the fifth most common cancer with about 74,000 new cases and 16,000 disease-specific deaths in 2015 in the United States (Siegel et al. (2015) Cancer Statistics 65(1):5-29). The majority of cases are non-muscle invasive bladder cancer (NMIBC) at diagnosis and are primarily managed with transurethral resection (TUR). With a recurrence rate of up to ˜70% at 5 years, bladder cancer requires lifelong cystoscopic surveillance (Aldousari et al. (2010) Can. Urol. Assoc. J. 4(1):56-64). Due to the invasiveness of cystoscopy, there are strong interests to develop non-invasive, urine-based diagnostics. A reliable urine test could improve surveillance strategies by prioritizing high-risk patients to undergo cystoscopy and biopsy, while reducing procedural frequency in low-risk patients. Despite inadequate sensitivity for both low grade (LG) tumors at ˜20% and high grade (HG) tumors at ˜80%, urine cytology is widely used due to high diagnostic specificity (>95%), resulting in high positive predictive values that may direct treatment for patients with positive cytology (Fantony et al. (2015) J. Natl. Compr. Canc. Ne. 13(9):1163-1166). Other FDA-approved urine tests including singleplex immunoassays, fluorescent immunohistochemistry, and fluorescence in-situ hybridization (Cheung et al. (2013) BMC Medicine 11:13; Breen et al. (2015) BMC Med. Res. Methodol. 15:45) are available, however, these tests have not been widely adopted due to insufficient diagnostic performance (Chang et al. (2016) J. Urol. 196(4): 1021-1029).

Emerging bladder cancer molecular diagnostics have focused on development of multi-biomarker panels ranging from 2 to 18 targets (Mengual et al. (2014) J. Urol. 191(1):261-269; O'Sullivan et al. (2012) J. Urol. 188(3):741-747; Holyoake et al. (2008) Clinical Cancer Research 14(3):742-749; Mengual et al. (2010) Clinical Cancer Research 16(9):2624-2633; Urquidi et al. (2016) Oncotarget 7(25):38731-38740). Most biomarker discovery efforts have depended on microarray-based screening of the bulk mass of tumor tissues. However, challenges of lower specificity than cytology and low sensitivity for LG tumors have remained (O'Sullivan et al., supra; Ribal et al. (2016) Eur. J. Cancer 54:131-138). To identify biomarkers for urine-based molecular diagnostics, exfoliated urothelial cells may be a better starting material given the continuous contact of bladder tumors with urine and their high translational potential (Street et al. (2014) J. Urol. 192(2):297-298).

RNA sequencing (RNA-seq) is a next generation sequencing technology that offers unbiased identification of known and novel transcripts, single base-pair resolution, high sensitivity and high specificity, broad dynamic range of over 8000-fold for gene expression quantification and ability to detect rare and low-abundance genes (Wang et al. (2009) Nat. Rev. Genet. 10(1):57-63).

There remains a need for sensitive and specific diagnostic tests for bladder cancer that can detect high-grade as well as low-grade bladder cancer.

### SUMMARY

The invention relates to the use of biomarkers for diagnosis of bladder cancer. In particular, the inventors have discovered biomarkers that can be used to diagnose bladder cancer, including determining whether an individual has high-grade bladder cancer or low-grade bladder cancer. These biomarkers can be used alone or in combination with one or more additional biomarkers or relevant clinical parameters in prognosis, diagnosis, or monitoring treatment of bladder cancer.

Biomarkers that can be used in the practice of the invention include polynucleotides comprising nucleotide sequences from genes or RNA transcripts of genes listed in Tables 4-10.

In certain embodiments, a panel of biomarkers is used for diagnosis of bladder cancer. Biomarker panels of any size can be used in the practice of the invention. Biomarker panels for diagnosing bladder cancer typically comprise at least 2 biomarkers and up to 30 biomarkers, including any number of biomarkers in between, such as 2, 3, 4, 5, 6, 7, 8, 9, 10, 11, 12, 13, 14, 15, 16, 17, 18, 19, 20, 21, 22, 23, 24, 25, 26, 27, 28, 29, or 30 biomarkers. In certain embodiments, the invention includes a biomarker panel comprising at least 2, at least 3, at least 4, or at least 5, or at least 6, or at least 7, or at least 8, or at least 9, or at least 10, or at least 11 or more biomarkers. Although smaller biomarker panels are usually more economical, larger biomarker panels (i.e., greater than 30 biomarkers) have the advantage of providing more detailed information and can also be used in the practice of the invention.

In certain embodiments, the invention includes a biomarker panel for diagnosing bladder cancer comprising at least two polynucleotides comprising nucleotide sequences from genes or RNA transcripts of genes selected from Tables 4-10. In one embodiment the biomarker panel comprises a ROBO1 polynucleotide and a WNT5A polynucleotide. In another embodiment, the biomarker panel further comprises one or more biomarkers selected from the group consisting of a RARRES1 polynucleotide, a CP polynucleotide, an IGFBP5 polynucleotide, a PLEKHS1 polynucleotide, a BPIFB1 polynucleotide, and a MYBPC1 polynucleotide. In another embodiment, the biomarker panel comprises a ROBO1 polynucleotide, a WNT5A polynucleotide, a RARRES1 polynucleotide, and a CP polynucleotide.

In another embodiment, the invention includes a biomarker panel for distinguishing low grade bladder cancer from high grade bladder cancer comprising one or more biomarkers selected from the group consisting of a MTRNR2L8 polynucleotide, a VEGFA polynucleotide, and an AKAP12 polynucleotide. In another embodiment, the biomarker panel comprises a MTRNR2L8 polynucleotide, a VEGFA polynucleotide, and an AKAP12 polynucleotide.

In another embodiment, the invention includes a method for diagnosing bladder cancer in a subject. The method comprises a) measuring the level of a plurality of biomarkers in a biological sample derived from the subject; and b) analyzing the level of expression of the plurality of biomarkers in conjunction with respective reference value ranges for said plurality of biomarkers, wherein differential expression of one or more biomarkers in the biological sample compared to reference value ranges of the biomarkers for a control subject indicate that the subject has bladder cancer. The reference value ranges can represent the levels of one or more biomarkers found in one or more samples of one or more subjects without bladder cancer (e.g., healthy subject or normal subject). Alternatively, the reference values can represent the levels of one or more biomarkers found in one or more samples of one or more subjects with bladder cancer. More specifically, the reference value ranges can represent the levels of one or more biomarkers at particular stages of disease (e.g., benign hyperplasia, low grade bladder cancer, or high grade bladder cancer) to facilitate a determination of the stage of disease progression in an individual and an appropriate treatment regimen.

In certain embodiments, the invention includes a method for diagnosing bladder cancer in a subject using a biomarker panel described herein. The method comprises: a) collecting a biological sample from the subject; b) measuring levels of expression of each biomarker of the biomarker panel in the biological sample; and c) comparing the levels of expression of each biomarker with respective reference value ranges for the biomarkers, wherein differential expression of the biomarkers of the biomarker panel in the biological sample compared to reference value ranges of the biomarkers for a control subject indicate that the subject has bladder cancer.

In another embodiment, the invention includes a method for diagnosing and treating bladder cancer in a subject, the method comprising: a) collecting a urine sample from the subject; b) isolating urinary cells from the urine sample; c) measuring levels of expression of ROBO1 and WNT5A biomarkers in the urinary cells; d) diagnosing the subject by analyzing the levels of expression of each biomarker in conjunction with respective reference value ranges for the biomarkers, wherein increased levels of expression of the ROBO1 and WNT5A biomarkers compared to the reference value ranges for the biomarkers for a control subject indicate that the subject has bladder cancer; and e) administering an anti-cancer treatment for the bladder cancer to the subject if the subject is diagnosed with bladder cancer, wherein the anti-cancer treatment comprises surgical removal of the bladder cancer, immunotherapy, or chemotherapy.

In another embodiment, the method further comprises removing white blood cells and red blood cells from the urine sample prior to isolating the urinary cells.

In another embodiment, the method further comprises measuring a level of expression of at least one reference marker selected from the group consisting of QRICH1, CDC42BPB and DNMBP, wherein the level of expression of at least one reference marker is used for data normalization in order to allow comparison of corresponding values for different datasets. Normalization is performed to eliminate differences between samples caused, for example, by differences in sample collection and processing in order to accurately determine relative biomarker expression levels for samples. The level of a reference marker can be used for normalization of data for multiple samples, for example, to allow comparison of levels of biomarkers in biological samples collected from a patient at different time points or to compare levels of biomarkers to reference value ranges for the biomarkers that are determined from control or reference samples.

In another embodiment, the method further comprises measuring levels of expression of one or more biomarkers selected from the group consisting of RARRES1, CP, IGFBP5, PLEKHS1, BPIFB1, and MYBPC1, wherein increased levels of expression of the ROBO1 and WNT5A biomarkers in combination with increased levels of expression of the one or more biomarkers selected from the group consisting of RARRES1, CP, IGFBP5, PLEKHS1, BPIFB1, and MYBPC1 compared to reference value ranges for the biomarkers for a control subject indicate that the subject has bladder cancer.

In another embodiment, the method further comprises measuring levels of expression of RARRES1 and CP, wherein increased levels of expression of the ROBO1 and WNT5A biomarkers in combination with increased levels of expression of the RARRES1 and CP biomarkers compared to reference value ranges for the biomarkers for a control subject indicate that the subject has bladder cancer.

In another embodiment, the method further comprises measuring levels of expression of one or more additional genes selected from Tables 4-10, wherein differential expression of the one or more additional genes compared to reference value ranges for the genes for a control subject indicate that the subject has bladder cancer.

In certain embodiments, the anti-cancer treatment comprises surgical removal of at least a portion of the bladder cancer, for example, by transurethral resection of a bladder tumor.

In certain embodiments, a subject diagnosed with bladder cancer by a method described herein may be administered (e.g., intravesicularly) a therapeutically effective amount of Bacillus Calmette-Guerin (BCG).

In other embodiments, a subject diagnosed with bladder cancer by a method described herein may be administered (e.g., intravesicularly) a therapeutically effective amount of a chemotherapeutic agent selected from the group consisting of mitomycin (e.g., intravesical mitomycin therapy or electromotive mitomycin therapy), valrubicin, docetaxel, thiotepa, and gemcitabine.

Methods of the invention, as described herein, can be used to distinguish a diagnosis of bladder cancer from benign hyperplasia and to determine the stage of cancer progression (e.g., high-grade or low-grade bladder cancer). In certain embodiments, the method comprises measuring levels of expression of one or more genes selected from Tables 5 and 6 in the urinary cells, and distinguishing whether the subject has low-grade bladder cancer or high-grade bladder cancer by comparing the levels of expression of the one or more genes selected from Tables 5 and 6 to reference value ranges for subjects having low-grade bladder cancer or high-grade bladder cancer. In one embodiment, the method comprises measuring levels of expression in the urinary cells of one or more genes selected from Table 5, wherein differential expression of the one or more genes selected from Tables 5 compared to reference value ranges for a control subject indicate that the subject has high grade bladder cancer. In another embodiment, the method comprises measuring levels of expression in the urinary cells of one or more genes selected from Table 6, wherein differential expression of the one or more genes selected from Tables 6 compared to reference value ranges for a control subject indicate that the subject has low grade bladder cancer. In another embodiment, the method comprises measuring levels of expression of one or more genes selected from the group consisting of MTRNR2L8, VEGFA, and AKAP12 in the urinary cells, wherein increased expression of the one or more genes selected from the group consisting of MTRNR2L8, VEGFA, and AKAP12 compared to reference value ranges for a subject having low grade bladder cancer indicates that the subject has high grade bladder cancer and decreased expression of the one or more genes selected from the group consisting of MTRNR2L8, VEGFA, and AKAP12 compared to reference value ranges for a subject having high grade bladder cancer indicates that the subject has low grade bladder cancer.

The biological sample may comprise, for example, urine, urothelial cells, or a biopsy from a bladder cancer. In particular, the biological sample may comprise cancerous cells from a bladder tumor that are exfoliated into the urine of a subject. Such cancerous cells may be isolated from samples of urine, for example, by centrifugation. In certain embodiments, blood cells, including red blood cells and white blood cells are removed from the biological sample prior to determining biomarker levels.

Biomarker polynucleotides (e.g., RNA transcripts) can be detected, for example, by microarray analysis, polymerase chain reaction (PCR), reverse transcriptase polymerase chain reaction (RT-PCR), Northern blot, or serial analysis of gene expression (SAGE).

In another aspect, the invention includes a method of performing endoscopy screening for bladder cancer, the method comprising: a) collecting a urine sample from the subject; b) isolating urinary cells from the urine sample; c) measuring levels of expression of one or more biomarkers, described herein, in the urinary cells; d) analyzing the levels of expression of each biomarker in conjunction with respective reference value ranges for the biomarkers, wherein differential expression of one or more biomarkers compared to the reference value ranges for the biomarkers for a control subject indicate that the subject has bladder cancer; and e) performing the endoscopy screening on the subject if the levels of expression of one or more biomarkers indicate that the subject has bladder cancer, or reducing the frequency of the endoscopy screening for bladder cancer if the levels of expression of one or more biomarkers indicate that the subject does not have bladder cancer.

In one embodiment, the method of performing endoscopy screening for bladder cancer comprises: a) collecting a urine sample from the subject; b) isolating urinary cells from the urine sample; c) measuring levels of expression of ROBO1 and WNT5A biomarkers in the urinary cells; d) analyzing the levels of expression of each biomarker in conjunction with respective reference value ranges for the biomarkers, wherein increased levels of expression of the ROBO1 and WNT5A biomarkers compared to the reference value ranges for the biomarkers for a control subject indicate that the subject has bladder cancer; and e) performing the endoscopy screening on the subject if the levels of expression of the ROBO1 and WNT5A biomarkers indicate that the subject has bladder cancer, or reducing the frequency of the endoscopy screening for bladder cancer if the levels of expression of the ROBO1 and WNT5A biomarkers indicate that the subject does not have bladder cancer.

In certain embodiments, reducing the frequency of the endoscopy screening comprises waiting to perform endoscopy screening until the levels of expression of the biomarkers indicate that the subject has bladder cancer. In other embodiments, reducing the frequency of endoscopy screening comprises performing endoscopy screening once a year, every other year, or every 2, 3, 4, or 5 years if the levels of expression of the biomarkers indicate that the subject does not have bladder cancer.

The methods described herein for prognosis or diagnosis of bladder cancer may be used in individuals who have not yet been diagnosed (for example, preventative screening), or who have been diagnosed, or who are suspected of having bladder cancer (e.g., display one or more characteristic symptoms), or who are at risk of developing bladder cancer (e.g., have a genetic predisposition or presence of one or more developmental, environmental, occupational, or behavioral risk factors). In particular, a subject may be at risk of having bladder cancer because of smoking, chronic catheterization, or an environmental exposure to a carcinogen. Subjects in certain occupations, such as, but not limited to, veterans, firefighters, chemists, bus drivers, rubber workers, mechanics, leather workers, blacksmiths, machine setters, or hairdressers may also be at higher risk of developing bladder cancer and benefit from diagnostic screening for bladder cancer by the methods described herein.

In another embodiment, the method further comprises measuring levels of expression of one or more biomarkers selected from the group consisting of RARRES1, CP, IGFBP5, PLEKHS1, BPIFB1, and MYBPC1, wherein increased levels of expression of the ROBO1 and WNT5A biomarkers in combination with increased levels of expression of the one or more biomarkers selected from the group consisting of RARRES1, CP, IGFBP5, PLEKHS1, BPIFB1, and MYBPC1 compared to reference value ranges for the biomarkers for a control subject indicate that the subject has bladder cancer; and performing the endoscopy screening on the subject if the levels of expression of the ROBO1 and WNT5A biomarkers in combination with the levels of expression of the one or more biomarkers selected from the group consisting of RARRES1, CP, IGFBP5, PLEKHS1, BPIFB1, and MYBPC1 indicate that the subject has bladder cancer, or reducing the frequency of the endoscopy screening for bladder cancer if the levels of expression of the ROBO1 and WNT5A biomarkers in combination with the levels of expression of the one or more biomarkers selected from the group consisting of RARRES1, CP, IGFBP5, PLEKHS1, BPIFB1, and MYBPC1 biomarkers indicate that the subject does not have bladder cancer.

In another embodiment, the method further comprises measuring levels of expression of RARRES1 and CP biomarkers, wherein increased levels of expression of the ROBO1 and WNT5A biomarkers in combination with increased levels of expression of the RARRES1 and CP biomarkers compared to reference value ranges for the biomarkers for a control subject indicate that the subject has bladder cancer; and performing the endoscopy screening on the subject if the levels of expression of the ROBO1, WNT5A, RARRES1 and CP biomarkers indicate that the subject has bladder cancer, or reducing the frequency of the endoscopy screening for bladder cancer if the levels of expression of the ROBO1, WNT5A, RARRES1 and CP biomarkers indicate that the subject does not have bladder cancer.

In another embodiment, the method further comprises measuring levels of expression of one or more additional genes selected from Tables 4-10 and analyzing the levels of expression of the one or more additional genes in conjunction with respective reference value ranges for the genes.

In another embodiment, the invention includes a method for evaluating the effect of an agent for treating bladder cancer in a subject, the method comprising: analyzing the levels of expression of one or more biomarkers described herein in samples derived from the subject before and after the subject is treated with the agent in conjunction with respective reference value ranges for the biomarkers.

In another embodiment, the invention includes a method for monitoring the efficacy of a therapy for treating bladder cancer in a subject, the method comprising: analyzing the levels of expression of one or more biomarkers described herein in samples derived from the subject before and after the subject undergoes the therapy in conjunction with respective reference value ranges for the biomarkers.

In another embodiment, the invention includes a method for monitoring the efficacy of a therapy for treating bladder cancer in a subject, the method comprising: measuring levels of expression of ROBO1 and WNT5A biomarkers in a first sample derived from the subject before the subject undergoes said therapy and a second sample derived from the subject after the subject undergoes said therapy, wherein increased levels of expression of the ROBO1 and WNT5A biomarkers in the second sample compared to the levels of expression of the biomarkers in the first sample indicate that the subject is worsening, and decreased levels of expression of the ROBO1 and WNT5A biomarkers in the second sample compared to the levels of expression of the biomarkers in the first sample indicate that the subject is improving. The method may further comprise measuring a level of expression of at least one reference marker selected from the group consisting of QRICH1, CDC42BPB and DNMBP, wherein the level of expression of the at least one reference marker is used for data normalization.

In another embodiment, the method further comprises measuring levels of expression of one or more biomarkers selected from the group consisting of RARRES1, CP, IGFBP5, PLEKHS1, BPIFB1, and MYBPC1 in the first sample derived from the subject before the subject undergoes said therapy and the second sample derived from the subject after the subject undergoes said therapy, wherein increased levels of expression of the ROBO1 and WNT5A biomarkers in combination with increased levels of expression of the one or more biomarkers selected from the group consisting of RARRES1, CP, IGFBP5, PLEKHS1, BPIFB1, and MYBPC1 in the second sample compared to the levels of expression of the biomarkers in the first sample indicate that the subject is worsening, and decreased levels of expression of the ROBO1 and WNT5A biomarkers in combination with decreased levels of expression of the one or more biomarkers selected from the group consisting of RARRES1, CP, IGFBP5, PLEKHS1, BPIFB1, and MYBPC1 in the second sample compared to the levels of expression of the biomarkers in the first sample indicate that the subject is improving.

In another embodiment, the method further comprises measuring levels of expression of RARRES1 and CP biomarkers in the first sample derived from the subject before the subject undergoes said therapy and the second sample derived from the subject after the subject undergoes said therapy, wherein increased levels of expression of the ROBO1 and WNT5A biomarkers in combination with increased levels of expression of the RARRES1 and CP biomarkers in the second sample compared to the levels of expression of the biomarkers in the first sample indicate that the subject is worsening, and decreased levels of expression of the ROBO1 and WNT5A biomarkers in combination with decreased levels of expression of the RARRES1 and CP biomarkers in the second sample compared to the levels of expression of the biomarkers in the first sample indicate that the subject is improving.

In another embodiment, the method further comprises measuring levels of expression of one or more additional genes selected from Tables 4-10 in samples derived from the subject before and after the subject undergoes the therapy, and analyzing the levels of expression of the genes in conjunction with respective reference value ranges for the genes.

In another embodiment, the invention includes a method for monitoring the efficacy of a therapy for treating bladder cancer in a subject, the method comprising: measuring levels of expression of MTRNR2L8, VEGFA, and AKAP12 biomarkers in a first sample derived from the subject before the subject undergoes said therapy and a second sample derived from the subject after the subject undergoes said therapy, wherein increased levels of expression of the MTRNR2L8, VEGFA, and AKAP12 biomarkers in the second sample compared to the levels of expression of the biomarkers in the first sample indicate that the subject is worsening, and decreased levels of expression of the MTRNR2L8, VEGFA, and AKAP12 biomarkers in the second sample compared to the levels of expression of the biomarkers in the first sample indicate that the subject is improving. The method may further comprise measuring a level of expression of at least one reference marker selected from the group consisting of QRICH1, CDC42BPB and DNMBP, wherein the level of expression of the at least one reference marker is used for data normalization.

In another embodiment, the method further comprises measuring levels of expression of one or more additional genes selected from Tables 4-10 in samples derived from the subject before and after the subject undergoes the therapy, and analyzing the levels of expression of the genes in conjunction with respective reference value ranges for the genes.

In another embodiment, the method further comprises measuring levels of expression of one or more biomarkers selected from the group consisting of ROBO1, WNT5A, RARRES1, CP, IGFBP5, PLEKHS1, BPIFB1, and MYBPC1 biomarkers in the first sample derived from the subject before the subject undergoes said therapy and the second sample derived from the subject after the subject undergoes said therapy, wherein increased levels of expression of the MTRNR2L8, VEGFA, and AKAP12 biomarkers in combination with increased levels of expression of the one or more biomarkers selected from the group consisting of ROBO1, WNT5A, RARRES1, CP, IGFBP5, PLEKHS1, BPIFB1, and MYBPC1 in the second sample compared to the levels of expression of the biomarkers in the first sample indicate that the subject is worsening, and decreased levels of expression of the MTRNR2L8, VEGFA, and AKAP12 biomarkers in combination with decreased levels of expression of the one or more biomarkers selected from the group consisting of ROBO1, WNT5A, RARRES1, CP, IGFBP5, PLEKHS1, BPIFB1, and MYBPC1 in the second sample compared to the levels of expression of the biomarkers in the first sample indicate that the subject is improving.

In another aspect, the invention includes a kit for diagnosing bladder cancer in a subject. The kit may include a container for holding a biological sample (e.g., urine, urine cells, or bladder cancer biopsy) isolated from a human subject suspected of having bladder cancer, at least one agent that specifically detects a bladder cancer biomarker; and printed instructions for reacting the agent with the biological sample or a portion of the biological sample to detect the presence or amount of at least one bladder cancer biomarker in the biological sample. The agents may be packaged in separate containers. The kit may further comprise one or more control reference samples and reagents for performing PCR or microarray analysis for detection of biomarkers as described herein. The kit may further comprise information, in electronic or paper form, comprising instructions to correlate the detected levels of each biomarker with bladder cancer.

In certain embodiments, the kit comprises agents for measuring the levels of expression of one or more genes selected from Tables 4-10.

In another embodiment, the kit further comprises at least one set of PCR primers capable of amplifying a nucleic acid comprising a sequence of a gene selected from Tables 4-10 or its complement.

In another embodiment, the kit further comprises at least one probe capable of hybridizing to a nucleic acid comprising a sequence of a gene selected from Table 4-10 or its complement.

In certain embodiments, the kit includes agents for detecting polynucleotides of a biomarker panel comprising a plurality of biomarkers for diagnosing bladder cancer, wherein one or more biomarkers are selected from the group consisting of a WNT5A polynucleotide, a RARRES1 polynucleotide, a ROBO1 polynucleotide, a CP polynucleotide, an IGFBP5 polynucleotide, a PLEKHS1 polynucleotide, a BPIFB1 polynucleotide, and a MYBPC1 polynucleotide.

In certain embodiments, the kit comprises agents for measuring the levels of expression of ROBO1 and WNT5A. In another embodiment, the kit further comprises at least one agent for measuring a level of expression of at least one reference marker selected from the group consisting of QRICH1, CDC42BPB and DNMBP. In another embodiment, the kit further comprises agents for measuring the levels of expression of one or more biomarkers selected from the group consisting of RARRES1, CP, IGFBP5, PLEKHS1, BPIFB1, and MYBPC1. In another embodiment, the kit comprises agents for measuring the levels of expression of RARRES1 and CP. In another embodiment, the kit further comprises agents for measuring the levels of expression of one or more additional genes selected from Tables 4-10.

In another embodiment, the kit comprises agents for measuring the levels of expression of one or more genes selected from the group consisting of MTRNR2L8, VEGFA, and AKAP12. In another embodiment, the kit comprises agents for measuring the levels of expression of MTRNR2L8, VEGFA, and AKAP12.

In another embodiment, the kit further comprises at least one set of PCR primers capable of amplifying a nucleic acid comprising a sequence of a gene selected from Table 5 or Table 6 or its complement.

In another embodiment, the kit further comprises at least one probe capable of hybridizing to a nucleic acid comprising a sequence of a gene selected from Table 5 or Table 6 or its complement.

In certain embodiments, the kit comprises a microarray comprising an oligonucleotide that hybridizes to a ROBO1 polynucleotide and an oligonucleotide that hybridizes to a WNT5A polynucleotide. In another embodiment, the microarray further comprises an oligonucleotide that hybridizes to a CDC42BPB polynucleotide.

In another embodiment, the microarray further comprises an oligonucleotide that hybridizes to a RARRES1 polynucleotide and an oligonucleotide that hybridizes to a CP polynucleotide.

In another embodiment, the microarray further comprises an oligonucleotide that hybridizes to a RARRES1 polynucleotide, an oligonucleotide that hybridizes to a CP polynucleotide, an oligonucleotide that hybridizes to an IGFBP5 polynucleotide, an oligonucleotide that hybridizes to a PLEKHS1 polynucleotide, an oligonucleotide that hybridizes to a BPIFB1 polynucleotide, and an oligonucleotide that hybridizes to a MYBPC1 polynucleotide.

In another embodiment, the microarray further comprises an oligonucleotide that hybridizes to a MTRNR2L8 polynucleotide, an oligonucleotide that hybridizes to a VEGFA polynucleotide, and an oligonucleotide that hybridizes to an AKAP12 polynucleotide.

In another aspect, the invention includes a method of distinguishing whether a subject has low-grade bladder cancer or high-grade bladder cancer and treating the subject for bladder cancer, the method comprising: a) collecting a urine sample from the subject; b) isolating urinary cells from the urine sample; c) measuring levels of expression of the one or more genes selected from the group consisting of MTRNR2L8, VEGFA, and AKAP12 in the urinary cells; d) distinguishing whether the subject has low-grade bladder cancer or high-grade bladder cancer by analyzing the levels of expression of the one or more genes selected from the group consisting of MTRNR2L8, VEGFA, and AKAP12 in conjunction with respective reference value ranges for subjects with low-grade bladder cancer or high-grade bladder cancer, wherein increased levels of expression of the one or more genes selected from the group consisting of MTRNR2L8, VEGFA, and AKAP12 compared to the reference value ranges for a subject having low grade bladder cancer indicate that the subject has high grade bladder cancer and decreased levels of expression of the one or more genes selected from the group consisting of MTRNR2L8, VEGFA, and AKAP12 compared to the reference value ranges for a subject having high grade bladder cancer indicate that the subject has low grade bladder cancer; and e) administering an anti-cancer treatment for high grade bladder cancer to the subject if the subject is diagnosed with high grade bladder cancer, and administering an anti-cancer treatment for low grade bladder cancer to the subject if the subject is diagnosed with low grade bladder cancer.

In certain embodiments, the method further comprises measuring levels of expression of one or more additional genes selected from Tables 5 and 6 in the urinary cells, and comparing the levels of expression of the one or more additional genes selected from Tables 5 and 6 to reference value ranges for subjects having low-grade bladder cancer or high-grade bladder cancer.

In another embodiment, the method comprises measuring levels of expression in the urinary cells of one or more genes selected from Table 5, wherein differential expression of the one or more genes selected from Tables 5 compared to reference value ranges for a control subject indicate that the subject has high grade bladder cancer.

In another embodiment, the method comprises measuring levels of expression in the urinary cells of one or more genes selected from Table 6, wherein differential expression of the one or more genes selected from Tables 6 compared to reference value ranges for a control subject indicate that the subject has low grade bladder cancer.

These and other embodiments of the subject invention will readily occur to those of skill in the art in view of the disclosure herein.

### BRIEF DESCRIPTION OF THE FIGURES

FIGS. 1A-1C show the approach for development and validation of a new urine test for bladder cancer. For the biomarker discovery in part 1 (FIG. 1A), urine samples from 13 bladder cancer patients and 10 control subjects were collected for RNA-seq analysis. For model construction in part 2 (FIG. 1B), a subset of genes that were differentially expressed in bladder cancer compared to controls was selected for qPCR validation in 102 urine samples. A model for computing a probability of bladder cancer score (P_{BC}) based on the gene expression of the 3-marker panel in urine was constructed using multivariate logistic regression. For model validation in part 3 (FIG. 1C), the diagnostic performance of the 3-marker panel was evaluated in an independent study cohort of 101 urine samples.

FIGS. 2A-2C show the diagnostic performance of the 3-marker panel for bladder cancer prediction. The probability of bladder cancer score (P_{BC}) based on the diagnostic equation using the 3-marker (ROBO1, WNT5A, CDC42BPB) urine assay was measured in FIG. 2A, the training cohort (n=102) and FIG. 2B, the validation cohort (n=101). P_{BC}≥0.45 (the black line in FIGS. 2A and 2B) as the threshold for a positive test gave the best concordance with clinical findings for patients without evidence of bladder cancer (Neg cysto, BC-evaluation; Neg cysto, BC-surveillance; Neg cysto, others (other non-neoplastic urological diseases); and Healthy controls) and patient with bladder cancer (HG and LG). FIG. 2C shows a comparison of the diagnostic performance of the 3-marker in the validation cohort (n=101) with cytology on a subset of samples (n=89) using ROC curves resulting in AUCs of 0.87 for the 3-marker panel and 0.68 for cytology. Neg cysto, Negative cystoscopy.

FIGS. 3A-3F show bladder cancer surveillance using the 3-marker urine test. Serial urine samples were collected from 6 patients and the probability of bladder cancer score (P_{BC}) based on the 3-marker (ROBO1, WNT5A, CDC42BPB) diagnostic equation was determined. P_{BC}≥0.45 (black line) was considered positive for bladder cancer. Corresponding bladder cancer pathology (stage, grade) or cystoscopy (if no bladder cancer detected) was indicated above urine test result. FIG. 3A shows that a urine test can accurately detect persistent bladder cancer. Test 1 for bladder cancer evaluation accurately detected bladder cancer as did follow up surveillance tests after 5 months (test 2) and another 6 months (test 3). FIG. 3B shows that a urine test can accurately detect bladder cancer recurrence in patient disease free for >16 months. Test 1 for bladder cancer surveillance was negative consistent with negative cystoscopy, as were tests 2 and 3 at 3 month intervals, test 4 accurately detected bladder cancer recurrence 10 months later. FIG. 3C shows that the urine test was reliable for prediction of alternating pattern of positive and negative tests. Test 1 for bladder cancer evaluation accurately detected bladder cancer. Follow up surveillance at 3 months was negative by both urine test and cystoscopy. Bladder cancer recurrence was accurately detected after another 9 months, followed by negative results from both urine test and cystoscopy after another 5 months.

FIGS. 3D, 3E and 3F show that after an initial positive bladder cancer test, the subsequent urine tests accurately predicted disease-free survival. Test 1 for bladder cancer surveillance (FIG. 3D) or bladder cancer evaluation (FIGS. 3E and 3F) accurately detected bladder cancer. Subsequent surveillance tests were negative by both urine test and cystoscopy (low grade (LG); high grade (HG)).

FIG. 4 shows gene expression of candidate reference genes for model construction. The gray dots represent the absolute Cₜ values for 29 urine samples assayed with the standard deviation plotted as the black error bar. The gray line indicates the grand mean Cₜ value of all samples over all 5 genes.

### DETAILED DESCRIPTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Bladder Cancer: Diagnosis, Therapeutics, and Management (Current Clinical Urology, C. T. Lee and D. P. Wood eds., Humana Press, 2010 edition); Bladder Cancer: Diagnosis and Clinical Management (S. P. Lerner, M. P. Schoenberg, and C. N. Sternberg eds., Wiley-Blackwell, 2015); Carcinoma of the Bladder (Progress in Cancer Research and Therapy Ser.: Vol. 18, J. G. Connolly ed., Raven Pr, 1981); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (3rd Edition, 2001); Methods In Enzymology (S. Colowick and N. Kaplan eds., Academic Press, Inc.).

All publications, patents and patent applications cited herein, whether supra or infra, are hereby incorporated by reference in their entireties.

### I. Definitions

In describing the present invention, the following terms will be employed, and are intended to be defined as indicated below.

It must be noted that, as used in this specification and the appended claims, the singular forms “a,” “an,” and “the” include plural referents unless the content clearly dictates otherwise. Thus, for example, reference to “a biomarker” includes a mixture of two or more biomarkers, and the like.

The term “about,” particularly in reference to a given quantity, is meant to encompass deviations of plus or minus five percent.

A “biomarker” in the context of the present invention refers to a biological compound, such as a polynucleotide or polypeptide which is differentially expressed in a sample taken from a patient having bladder cancer (e.g., urine sample containing cancerous urothelial cells) as compared to a comparable sample taken from a control subject (e.g., a person with a negative diagnosis, normal or healthy subject, or subject without bladder cancer). The biomarker can be a nucleic acid, a fragment of a nucleic acid, a polynucleotide, or an oligonucleotide that can be detected and/or quantified. Bladder cancer biomarkers include polynucleotides comprising nucleotide sequences from genes or RNA transcripts of genes, including but not limited to, the genes listed in Tables 4-10.

The terms “polypeptide” and “protein” refer to a polymer of amino acid residues and are not limited to a minimum length. Thus, peptides, oligopeptides, dimers, multimers, and the like, are included within the definition. Both full-length proteins and fragments thereof are encompassed by the definition. The terms also include postexpression modifications of the polypeptide, for example, glycosylation, acetylation, phosphorylation, hydroxylation, oxidation, and the like.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), and any other type of polynucleotide which is an N- or C-glycoside of a purine or pyrimidine base. There is no intended distinction in length between the terms “polynucleotide,” “oligonucleotide,” “nucleic acid” and “nucleic acid molecule,” and these terms are used interchangeably.

The phrase “differentially expressed” refers to differences in the quantity and/or the frequency of a biomarker present in a sample taken from patients having, for example, bladder cancer as compared to a control subject or subject without cancer. For example, a biomarker can be a polynucleotide which is present at an elevated level or at a decreased level in samples of patients with bladder cancer compared to samples of control subjects. Alternatively, a biomarker can be a polynucleotide which is detected at a higher frequency or at a lower frequency in samples of patients with bladder cancer compared to samples of control subjects. A biomarker can be differentially present in terms of quantity, frequency or both.

A polynucleotide is differentially expressed between two samples if the amount of the polynucleotide in one sample is statistically significantly different from the amount of the polynucleotide in the other sample. For example, a polynucleotide is differentially expressed in two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polynucleotide is differentially expressed in two sets of samples if the frequency of detecting the polynucleotide in samples of patients' suffering from bladder cancer, is statistically significantly higher or lower than in the control samples. For example, a polynucleotide is differentially expressed in two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

A “similarity value” is a number that represents the degree of similarity between two things being compared. For example, a similarity value may be a number that indicates the overall similarity between a patient's expression profile using specific phenotype-related biomarkers and reference value ranges for the biomarkers in one or more control samples or a reference expression profile (e.g., the similarity to a “bladder cancer” expression profile, a “high grade bladder cancer” expression profile, or a “low grade bladder cancer” expression profile). The similarity value may be expressed as a similarity metric, such as a correlation coefficient, or may simply be expressed as the expression level difference, or the aggregate of the expression level differences, between levels of biomarkers in a patient sample and a control sample or reference expression profile.

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; and primates.

As used herein, a “biological sample” refers to a sample of tissue, cells, or fluid isolated from a subject, including but not limited to, for example, urine, urothelial cells, a bladder cancer biopsy, blood, buffy coat, plasma, serum, blood cells (e.g., peripheral blood mononucleated cells (PBMCS), band cells, neutrophils, monocytes, or T cells), fecal matter, bone marrow, bile, spinal fluid, lymph fluid, samples of the skin, external secretions of the skin, respiratory, intestinal, and genitourinary tracts, tears, saliva, milk, organs, biopsies and also samples of in vitro cell culture constituents, including, but not limited to, conditioned media resulting from the growth of cells and tissues in culture medium, e.g., recombinant cells, and cell components.

A “test amount” of a biomarker refers to an amount of a biomarker present in a sample being tested. A test amount can be either an absolute amount (e.g., μg/ml) or a relative amount (e.g., relative intensity of signals).

A “diagnostic amount” of a biomarker refers to an amount of a biomarker in a subject's sample that is consistent with a diagnosis of bladder cancer. A diagnostic amount can be either an absolute amount (e.g., μg/ml) or a relative amount (e.g., relative intensity of signals).

A “control amount” of a biomarker can be any amount or a range of amount which is to be compared against a test amount of a biomarker. For example, a control amount of a biomarker can be the amount of a biomarker in a person without bladder cancer. A control amount can be either in absolute amount (e.g., μg/ml) or a relative amount (e.g., relative intensity of signals).

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

“Detectable moieties” or “detectable labels” contemplated for use in the invention include, but are not limited to, radioisotopes, fluorescent dyes such as fluorescein, phycoerythrin, Cy-3, Cy-5, allophycoyanin, DAPI, Texas Red, rhodamine, Oregon green, Lucifer yellow, and the like, green fluorescent protein (GFP), red fluorescent protein (DsRed), cyan fluorescent Protein (CFP), yellow fluorescent protein (YFP), cerianthus orange fluorescent protein (cOFP), alkaline phosphatase (AP), beta-lactamase, chloramphenicol acetyltransferase (CAT), adenosine deaminase (ADA), aminoglycoside phosphotransferase (neo^{r}, G418^{r}) dihydrofolate reductase (DHFR), hygromycin-B-phosphotransferase (HPH), thymidine kinase (TK), lacZ (encoding β-galactosidase), and xanthine guanine phosphoribosyltransferase (XGPRT), beta-glucuronidase (gus), placental alkaline phosphatase (PLAP), secreted embryonic alkaline phosphatase (SEAP), or firefly or bacterial luciferase (LUC). Enzyme tags are used with their cognate substrate. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), and glass microparticles with digital holographic code images (see e.g., CyVera microbeads produced by Illumina (San Diego, Calif.). As with many of the standard procedures associated with the practice of the invention, skilled artisans will be aware of additional labels that can be used.

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

“Substantially purified” refers to nucleic acid molecules or proteins that are removed from their natural environment and are isolated or separated, and are at least about 60% free, preferably about 75% free, and most preferably about 90% free, from other components with which they are naturally associated.

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, III, IV or V) or grade (G1, G2, G3, etc.) of neoplasia, tumor, or cancer, or a neoplasia, tumor, cancer or metastasis that is progressing, worsening, stabilized or in remission. In particular, the terms “tumor,” “cancer” and “neoplasia” include carcinomas, such as squamous cell carcinoma, adenocarcinoma, adenosquamous carcinoma, anaplastic carcinoma, large cell carcinoma, and small cell carcinoma.

### II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The invention relates to the use of biomarkers either alone or in combination with clinical parameters for diagnosis of bladder cancer. In particular, the inventors have discovered biomarkers whose expression profile can be used to diagnose bladder cancer and to determine whether an individual has high grade or low grade bladder cancer (see Example 1).

In order to further an understanding of the invention, a more detailed discussion is provided below regarding the identified biomarkers and methods of using them in prognosis, diagnosis, or monitoring treatment of bladder cancer.

A. Biomarkers

Biomarkers that can be used in the practice of the invention include polynucleotides comprising nucleotide sequences from genes or RNA transcripts of genes listed in Tables 4-10. Differential expression of these biomarkers is associated with bladder cancer and therefore expression profiles of these biomarkers are useful for diagnosing bladder cancer.

Accordingly, in one aspect, the invention provides a method for diagnosing bladder cancer in a subject, comprising measuring the level of a plurality of biomarkers in a biological sample derived from a subject suspected of having bladder cancer, and analyzing the levels of the biomarkers and comparing with respective reference value ranges for the biomarkers, wherein differential expression of one or more biomarkers in the biological sample compared to one or more biomarkers in a control sample indicates that the subject has bladder cancer.

When analyzing the levels of biomarkers in a biological sample, the reference value ranges used for comparison can represent the levels of one or more biomarkers found in one or more samples of one or more subjects without bladder cancer (i.e., normal or control samples). Alternatively, the reference values can represent the levels of one or more biomarkers found in one or more samples of one or more subjects with bladder cancer. More specifically, the reference value ranges can represent the levels of one or more biomarkers at particular stages of disease (e.g., benign hyperplasia, low grade bladder cancer, or high grade bladder cancer) to facilitate a determination of the stage of disease progression in an individual and an appropriate treatment regimen.

In certain embodiments, the method further comprises measuring a level of expression of at least one reference marker selected from the group consisting of QRICH1, CDC42BPB and DNMBP, wherein the level of expression of at least one reference marker is used for data normalization in order to allow comparison of corresponding values for different datasets. Normalization is performed to eliminate differences between samples caused, for example, by differences in sample collection and processing in order to accurately determine relative biomarker expression levels for samples. The level of a reference marker can be used for normalization of data for multiple samples, for example, to allow comparison of levels of biomarkers in biological samples collected from a patient at different time points or to compare levels of biomarkers to reference value ranges for the biomarkers that are determined from control or reference samples.

The biological sample obtained from the subject to be diagnosed is typically urine, urothelial cells, or a bladder cancer biopsy, but can be any sample from bodily fluids, tissue or cells that contain the expressed biomarkers. A “control” sample, as used herein, refers to a biological sample, such as a bodily fluid, tissue, or cells that are not diseased. That is, a control sample is obtained from a normal or healthy subject (e.g. an individual known to not have bladder cancer). A biological sample can be obtained from a subject by conventional techniques. For example, urine can be spontaneously voided by a subject or collected by bladder catheterization. Urinary cells can be collected from urine by using centrifugation to sediment cells and then discarding urinary fluid. In addition, urothelial cells may be separated from blood cells (e.g. white blood cells and red blood cells) in urine by fluorescence-activated cell sorting (FACS) or magnetic-activated cell sorting (MACS), or any other cell sorting method known in the art.

In certain embodiments, the biological sample is a bladder tumor sample, including the entire tumor or a portion, piece, part, segment, or fraction of a tumor. Solid tissue samples can be obtained by surgical techniques according to methods well known in the art. A bladder cancer biopsy may be obtained by methods including, but not limited to, an aspiration biopsy, a brush biopsy, a surface biopsy, a needle biopsy, a punch biopsy, an excision biopsy, an open biopsy, an incision biopsy or an endoscopic biopsy.

In certain embodiments, a panel of biomarkers is used for diagnosis of bladder cancer. Biomarker panels of any size can be used in the practice of the invention. Biomarker panels for diagnosing bladder cancer typically comprise at least 2 biomarkers and up to 30 biomarkers, including any number of biomarkers in between, such as 2, 3, 4, 5, 6, 7, 8, 9, 10, 11, 12, 13, 14, 15, 16, 17, 18, 19, 20, 21, 22, 23, 24, 25, 26, 27, 28, 29, or 30 biomarkers. In certain embodiments, the invention includes a biomarker panel comprising at least 2, at least 3, or at least 4, or at least 5, or at least 6, or at least 7, or at least 8, or at least 9, or at least 10, or at least 11 or more biomarkers. Although smaller biomarker panels are usually more economical, larger biomarker panels (i.e., greater than 30 biomarkers) have the advantage of providing more detailed information and can also be used in the practice of the invention.

In certain embodiments, the invention includes a biomarker panel for diagnosing bladder cancer comprising at least two polynucleotides comprising nucleotide sequences from genes or RNA transcripts of genes selected from Tables 4-10. In one embodiment the biomarker panel comprises a ROBO1 polynucleotide and a WNT5A polynucleotide. In another embodiment, the biomarker panel further comprises one or more biomarkers selected from the group consisting of a RARRES1 polynucleotide, a CP polynucleotide, an IGFBP5 polynucleotide, a PLEKHS1 polynucleotide, a BPIFB1 polynucleotide, and a MYBPC1 polynucleotide. In another embodiment, the biomarker panel comprises a ROBO1 polynucleotide, a WNT5A polynucleotide, a RARRES1 polynucleotide, and a CP polynucleotide.

In another embodiment, the invention includes a biomarker panel for distinguishing low grade bladder cancer from high grade bladder cancer comprising one or more biomarkers selected from the group consisting of a MTRNR2L8 polynucleotide, a VEGFA polynucleotide, and an AKAP12 polynucleotide. In another embodiment, the biomarker panel comprises a MTRNR2L8 polynucleotide, a VEGFA polynucleotide, and an AKAP12 polynucleotide.

In another embodiment, the invention includes a method for diagnosing and treating bladder cancer in a subject, the method comprising: a) collecting a urine sample from the subject; b) isolating urinary cells from the urine sample; c) measuring levels of expression of ROBO1 and WNT5A biomarkers in the urinary cells; d) diagnosing the subject by analyzing the levels of expression of each biomarker in conjunction with respective reference value ranges for the biomarkers, wherein increased levels of expression of the ROBO1 and WNT5A biomarkers compared to the reference value ranges for the biomarkers for a control subject indicate that the subject has bladder cancer; and e) administering an anti-cancer treatment for the bladder cancer to the subject if the subject is diagnosed with bladder cancer, wherein the anti-cancer treatment comprises surgical removal of the bladder cancer, immunotherapy, or chemotherapy.

In another embodiment, the method further comprises measuring a level of expression of at least one reference marker selected from the group consisting of QRICH1, CDC42BPB and DNMBP, wherein the level of expression of at least one reference marker is used for data normalization in order to allow comparison of corresponding values for different datasets.

In another embodiment, the method further comprises measuring levels of expression of one or more biomarkers selected from the group consisting of RARRES1, CP, IGFBP5, PLEKHS1, BPIFB1, and MYBPC1, wherein increased levels of expression of the ROBO1 and WNT5A biomarkers in combination with increased levels of expression of the one or more biomarkers selected from the group consisting of RARRES1, CP, IGFBP5, PLEKHS1, BPIFB1, and MYBPC1 compared to reference value ranges for the biomarkers for a control subject indicate that the subject has bladder cancer.

In another embodiment, the method further comprises measuring levels of expression of RARRES1 and CP, wherein increased levels of expression of the ROBO1 and WNT5A biomarkers in combination with increased levels of expression of the RARRES1 and CP biomarkers compared to reference value ranges for the biomarkers for a control subject indicate that the subject has bladder cancer.

In another embodiment, the method further comprises measuring levels of expression of one or more additional genes selected from Tables 4-10, wherein differential expression of the one or more additional genes compared to reference value ranges for the levels of expression of the genes for a control subject indicate that the subject has bladder cancer.

The methods described herein may be used to determine if a patient should be treated for bladder cancer. For example, anti-cancer therapy is administered to a patient found to have a positive bladder cancer diagnosis based on a biomarker expression profile, as described herein. Anti-cancer therapy may comprise one or more of surgery, intravesical therapy, chemotherapy, immunotherapy, or biologic therapy. For example, bladder cancer may be treated by surgical removal of at least a portion of the bladder cancer by transurethral resection or cystectomy. Alternatively or additionally, a patient diagnosed with bladder cancer may be administered (e.g., using intravesical or electromotive therapy) a therapeutically effective amount of an immunotherapeutic agent, such as BCG, and/or a chemotherapeutic agent, such as mitomycin, valrubicin, docetaxel, thiotepa, or gemcitabine. Patients diagnosed with high-grade bladder cancer may be treated more aggressively than patients diagnosed with low-grade bladder cancer. For example, patients diagnosed with high-grade bladder cancer may be treated with more radical surgery (e.g., a cystectomy (removal of the bladder) rather than more limited tumor resection) and/or administering higher doses and/or more extended immunotherapy or chemotherapy than patients diagnosed with low-grade bladder cancer. See, e.g., Bladder Cancer: Diagnosis, Therapeutics, and Management (Current Clinical Urology, C. T. Lee and D. P. Wood eds., Humana Press, 2010 edition) and Bladder Cancer: Diagnosis and Clinical Management (S. P. Lerner, M. P. Schoenberg, and C. N. Sternberg eds., Wiley-Blackwell, 2015); herein incorporated by reference.

In one embodiment, the invention includes a method of treating a subject having bladder cancer, the method comprising: a) diagnosing the subject with bladder cancer according to a method described herein; and b) administering anti-cancer therapy to the subject if the patient has a positive diagnosis for bladder cancer.

In another embodiment, the invention includes a method of treating a subject suspected of having bladder cancer, the method comprising: a) receiving information regarding the diagnosis of the subject according to a method described herein; and b) administering anti-cancer therapy to the subject if the patient has a positive diagnosis for bladder cancer.

The methods of the invention, as described herein, can also be used for determining the prognosis of a subject and for monitoring treatment of a subject having bladder cancer. The inventors have shown that differential expression of the biomarkers listed in Tables 5-7 is correlated with the severity of bladder cancer (e.g., low-grade or high grade bladder cancer). For Example, higher levels of expression of MTRNR2L8, VEGFA, and AKAP12 are correlated with more aggressive disease (see Example 1 and Table 7).

Thus, a medical practitioner can monitor the progress of disease by measuring the levels of expression of one or more of the MTRNR2L8, VEGFA, and AKAP12 biomarkers in a biological sample from the patient. For example, decreased levels of expression of MTRNR2L8, VEGFA, and AKAP12 as compared to prior levels of expression (e.g., in a prior urine sample or bladder cancer biopsy) indicate the disease or condition in the subject is improving or has improved, whereas increased levels of expression of MTRNR2L8, VEGFA, and AKAP12 as compared to prior levels of expression indicates the disease or condition in the subject has worsened or is worsening. Such worsening could possibly result in cancer progression (e.g. from low grade to high grade bladder cancer), tumor growth, or metastasis.

The methods described herein for prognosis or diagnosis of bladder cancer may be used in individuals who have not yet been diagnosed (for example, preventative screening), or who have been diagnosed, or who are suspected of having bladder cancer (e.g., display one or more characteristic symptoms), or who are at risk of developing bladder cancer (e.g., have a genetic predisposition or presence of one or more developmental, environmental, or behavioral risk factors). In particular, a subject may be at risk of having bladder cancer because of smoking, chronic catheterization, or an environmental exposure to a carcinogen. Subjects in certain occupations, such as, but not limited to, veterans, firefighters, chemists, bus drivers, rubber workers, mechanics, leather workers, blacksmiths, machine setters, or hairdressers may also be at higher risk of developing bladder cancer and benefit from diagnostic screening for bladder cancer by the methods described herein.

The methods may also be used to detect various stages of progression or severity of disease (e.g., benign hyperplasia, low grade bladder cancer, or high grade bladder cancer). The methods may also be used to detect the response of disease to prophylactic or therapeutic treatments or other interventions. The methods can furthermore be used to help the medical practitioner in determining prognosis (e.g., worsening, status-quo, partial recovery, or complete recovery) of the patient, and the appropriate course of action, resulting in either further treatment or observation, or in discharge of the patient from the medical care center.

In addition, the methods of the invention can also be used in evaluating the need for endoscopy screening of subjects at risk of having bladder cancer. For example, the frequency of endoscopy screening for bladder cancer may be reduced if the levels of expression of one or more of the WNT5A, RARRES1, ROBO1, CP, IGFBP5, PLEKHS1, BPIFB1, and MYBPC1 biomarkers indicate that the subject does not have bladder cancer. In certain embodiments, reducing the frequency of endoscopy screening comprises performing endoscopy screening once a year, every other year, or every 2, 3, 4, or 5 years if the levels of expression of the biomarkers compared to the reference value ranges for the biomarkers indicate that the subject does not have bladder cancer. In another embodiment, reducing the frequency of the endoscopy screening comprises waiting to perform endoscopy screening until the levels of expression of the biomarkers indicate that the subject has bladder cancer.

In certain embodiments, the invention includes a method of performing endoscopy screening for bladder cancer, the method comprising: a) collecting a urine sample from the subject; b) isolating urinary cells from the urine sample; c) measuring levels of expression of ROBO1 and WNT5A biomarkers in the urinary cells; d) analyzing the levels of expression of each biomarker in conjunction with respective reference value ranges for the biomarkers, wherein increased levels of expression of the ROBO1 and WNT5A biomarkers compared to the reference value ranges for the biomarkers for a control subject indicate that the subject has bladder cancer; and e) performing the endoscopy screening on the subject if the levels of expression of the ROBO1 and WNT5A biomarkers indicate that the subject has bladder cancer, or reducing the frequency of the endoscopy screening for bladder cancer if the levels of expression of the ROBO1 and WNT5A biomarkers indicate that the subject does not have bladder cancer.

In another embodiment, the method of performing endoscopy screening for bladder cancer further comprises measuring levels of expression of one or more biomarkers selected from the group consisting of RARRES1, CP, IGFBP5, PLEKHS1, BPIFB1, and MYBPC1, wherein increased levels of expression of the ROBO1 and WNT5A biomarkers in combination with increased levels of expression of the one or more biomarkers selected from the group consisting of RARRES1, CP, IGFBP5, PLEKHS1, BPIFB1, and MYBPC1 compared to reference value ranges for the biomarkers for a control subject indicate that the subject has bladder cancer; and performing the endoscopy screening on the subject if the levels of expression of the ROBO1 and WNT5A biomarkers in combination with the levels of expression of the one or more biomarkers selected from the group consisting of RARRES1, CP, IGFBP5, PLEKHS1, BPIFB1, and MYBPC1 indicate that the subject has bladder cancer, or reducing the frequency of the endoscopy screening for bladder cancer if the levels of expression of the ROBO1 and WNT5A biomarkers in combination with the levels of expression of the one or more biomarkers selected from the group consisting of RARRES1, CP, IGFBP5, PLEKHS1, BPIFB1, and MYBPC1 biomarkers indicate that the subject does not have bladder cancer.

In another embodiment, the method of performing endoscopy screening for bladder cancer further comprises measuring levels of expression of RARRES1 and CP biomarkers, wherein increased levels of expression of the ROBO1 and WNT5A biomarkers in combination with increased levels of expression of the RARRES1 and CP biomarkers compared to reference value ranges for the biomarkers for a control subject indicate that the subject has bladder cancer; and performing the endoscopy screening on the subject if the levels of expression of the ROBO1, WNT5A, RARRES1 and CP biomarkers indicate that the subject has bladder cancer, or reducing the frequency of the endoscopy screening for bladder cancer if the levels of expression of the ROBO1, WNT5A, RARRES1 and CP biomarkers indicate that the subject does not have bladder cancer.

In another embodiment, the method of performing endoscopy screening for bladder cancer further comprises measuring a level of expression of at least one reference marker selected from the group consisting of QRICH1, CDC42BPB and DNMBP, wherein the level of expression of at least one reference marker is used for data normalization in order to allow comparison of corresponding values for different datasets.

In another embodiment, the method further comprises measuring levels of expression of one or more additional genes selected from Tables 4-10 and analyzing the levels of expression of the genes in conjunction with respective reference value ranges for the genes.

B. Detecting and Measuring Biomarkers

It is understood that the biomarkers in a sample can be measured by any suitable method known in the art. Measurement of the expression level of a biomarker can be direct or indirect. For example, the abundance levels of RNAs or proteins can be directly quantitated. Alternatively, the amount of a biomarker can be determined indirectly by measuring abundance levels of cDNAs, amplified RNAs or DNAs, or by measuring quantities or activities of RNAs, proteins, or other molecules (e.g., metabolites) that are indicative of the expression level of the biomarker. The methods for measuring biomarkers in a sample have many applications. For example, one or more biomarkers can be measured to aid in the diagnosis of bladder cancer, to determine the appropriate treatment for a subject, to monitor responses in a subject to treatment, or to identify therapeutic compounds that modulate expression of the biomarkers in vivo or in vitro.

Detecting Biomarker Polynucleotides

In one embodiment, the expression levels of the biomarkers are determined by measuring polynucleotide levels of the biomarkers. The levels of transcripts of specific biomarker genes can be determined from the amount of mRNA, or polynucleotides derived therefrom, present in a biological sample. Polynucleotides can be detected and quantitated by a variety of methods including, but not limited to, microarray analysis, polymerase chain reaction (PCR), reverse transcriptase polymerase chain reaction (RT-PCR), Northern blot, and serial analysis of gene expression (SAGE). See, e.g., Draghici Data Analysis Tools for DNA Microarrays, Chapman and Hall/CRC, 2003; Simon et al. Design and Analysis of DNA Microarray Investigations, Springer, 2004; Real-Time PCR: Current Technology and Applications, Logan, Edwards, and Saunders eds., Caister Academic Press, 2009; Bustin A-Z of Quantitative PCR (IUL Biotechnology, No. 5), International University Line, 2004; Velculescu et al. (1995) Science 270: 484-487; Matsumura et al. (2005) Cell. Microbiol. 7: 11-18; Serial Analysis of Gene Expression (SAGE): Methods and Protocols (Methods in Molecular Biology), Humana Press, 2008; herein incorporated by reference in their entireties.

In one embodiment, microarrays are used to measure the levels of biomarkers. An advantage of microarray analysis is that the expression of each of the biomarkers can be measured simultaneously, and microarrays can be specifically designed to provide a diagnostic expression profile for a particular disease or condition (e.g., bladder cancer).

Microarrays are prepared by selecting probes which comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

Probes used in the methods of the invention are preferably immobilized to a solid support which may be either porous or non-porous. For example, the probes may be polynucleotide sequences which are attached to a nitrocellulose or nylon membrane or filter covalently at either the 3′ or the 5′ end of the polynucleotide. Such hybridization probes are well known in the art (see, e.g., Sambrook, et al., Molecular Cloning: A Laboratory Manual (3rd Edition, 2001). Alternatively, the solid support or surface may be a glass or plastic surface. In one embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In one embodiment, the microarray comprises a support or surface with an ordered array of binding (e.g., hybridization) sites or “probes” each representing one of the biomarkers described herein. Preferably the microarrays are addressable arrays, and more preferably positionally addressable arrays. More specifically, each probe of the array is preferably located at a known, predetermined position on the solid support such that the identity (i.e., the sequence) of each probe can be determined from its position in the array (i.e., on the support or surface). Each probe is preferably covalently attached to the solid support at a single site.

Microarrays can be made in a number of ways, of which several are described below. However they are produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. Microarrays are generally small, e.g., between 1 cm² and 25 cm²; however, larger arrays may also be used, e.g., in screening arrays. Preferably, a given binding site or unique set of binding sites in the microarray will specifically bind (e.g., hybridize) to the product of a single gene in a cell (e.g., to a specific mRNA, or to a specific cDNA derived therefrom). However, in general, other related or similar sequences will cross hybridize to a given binding site.

As noted above, the “probe” to which a particular polynucleotide molecule specifically hybridizes contains a complementary polynucleotide sequence. The probes of the microarray typically consist of nucleotide sequences of no more than 1,000 nucleotides. In some embodiments, the probes of the array consist of nucleotide sequences of 10 to 1,000 nucleotides. In one embodiment, the nucleotide sequences of the probes are in the range of 10-200 nucleotides in length and are genomic sequences of one species of organism, such that a plurality of different probes is present, with sequences complementary and thus capable of hybridizing to the genome of such a species of organism, sequentially tiled across all or a portion of the genome. In other embodiments, the probes are in the range of 10-30 nucleotides in length, in the range of 10-40 nucleotides in length, in the range of 20-50 nucleotides in length, in the range of 40-80 nucleotides in length, in the range of 50-150 nucleotides in length, in the range of 80-120 nucleotides in length, or are 60 nucleotides in length.

The probes may comprise DNA or DNA “mimics” (e.g., derivatives and analogues) corresponding to a portion of an organism's genome. In another embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone (e.g., phosphorothioates).

DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). Typically, each probe on the microarray will be between 10 bases and 50,000 bases, usually between 300 bases and 1,000 bases in length. PCR methods are well known in the art, and are described, for example, in Innis et al., eds., PCR Protocols: A Guide To Methods And Applications, Academic Press Inc., San Diego, Calif. (1990); herein incorporated by reference in its entirety. It will be apparent to one skilled in the art that controlled robotic systems are useful for isolating and amplifying nucleic acids.

An alternative, preferred means for generating polynucleotide probes is by synthesis of synthetic polynucleotides or oligonucleotides, e.g., using N-phosphonate or phosphoramidite chemistries (Froehler et al., Nucleic Acid Res. 14:5399-5407 (1986); McBride et al., Tetrahedron Lett. 24:246-248 (1983)). Synthetic sequences are typically between about 10 and about 500 bases in length, more typically between about 20 and about 100 bases, and most preferably between about 40 and about 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., Nature 363:566-568 (1993); U.S. Pat. No. 5,539,083).

Probes are preferably selected using an algorithm that takes into account binding energies, base composition, sequence complexity, cross-hybridization binding energies, and secondary structure. See Friend et al., International Patent Publication WO 01/05935, published Jan. 25, 2001; Hughes et al., Nat. Biotech. 19:342-7 (2001).

A skilled artisan will also appreciate that positive control probes, e.g., probes known to be complementary and hybridizable to sequences in the target polynucleotide molecules, and negative control probes, e.g., probes known to not be complementary and hybridizable to sequences in the target polynucleotide molecules, should be included on the array. In one embodiment, positive controls are synthesized along the perimeter of the array. In another embodiment, positive controls are synthesized in diagonal stripes across the array. In still another embodiment, the reverse complement for each probe is synthesized next to the position of the probe to serve as a negative control. In yet another embodiment, sequences from other species of organism are used as negative controls or as “spike-in” controls.

The probes are attached to a solid support or surface, which may be made, e.g., from glass, plastic (e.g., polypropylene, nylon), polyacrylamide, nitrocellulose, gel, or other porous or nonporous material. One method for attaching nucleic acids to a surface is by printing on glass plates, as is described generally by Schena et al, Science 270:467-470 (1995). This method is especially useful for preparing microarrays of cDNA (See also, DeRisi et al, Nature Genetics 14:457-460 (1996); Shalon et al., Genome Res. 6:639-645 (1996); and Schena et al., Proc. Natl. Acad. Sci. U.S.A. 93:10539-11286 (1995); herein incorporated by reference in their entireties).

A second method for making microarrays produces high-density oligonucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270; herein incorporated by reference in their entireties) or other methods for rapid synthesis and deposition of defined oligonucleotides (Blanchard et al., Biosensors & Bioelectronics 11:687-690; herein incorporated by reference in its entirety). When these methods are used, oligonucleotides (e.g., 60-mers) of known sequence are synthesized directly on a surface such as a derivatized glass slide. Usually, the array produced is redundant, with several oligonucleotide molecules per RNA.

Other methods for making microarrays, e.g., by masking (Maskos and Southern, 1992, Nuc. Acids Res. 20:1679-1684; herein incorporated by reference in its entirety), may also be used. In principle, any type of array, for example, dot blots on a nylon hybridization membrane (see Sambrook, et al., Molecular Cloning: A Laboratory Manual, 3rd Edition, 2001) could be used. However, as will be recognized by those skilled in the art, very small arrays will frequently be preferred because hybridization volumes will be smaller.

Microarrays can also be manufactured by means of an ink jet printing device for oligonucleotide synthesis, e.g., using the methods and systems described by Blanchard in U.S. Pat. No. 6,028,189; Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 111-123; herein incorporated by reference in their entireties. Specifically, the oligonucleotide probes in such microarrays are synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Microarrays manufactured by this ink-jet method are typically of high density, preferably having a density of at least about 2,500 different probes per 1 cm². The polynucleotide probes are attached to the support covalently at either the 3′ or the 5′ end of the polynucleotide.

Biomarker polynucleotides which may be measured by microarray analysis can be expressed RNA or a nucleic acid derived therefrom (e.g., cDNA or amplified RNA derived from cDNA that incorporates an RNA polymerase promoter), including naturally occurring nucleic acid molecules, as well as synthetic nucleic acid molecules. In one embodiment, the target polynucleotide molecules comprise RNA, including, but by no means limited to, total cellular RNA, poly(A)⁺ messenger RNA (mRNA) or a fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. Nos. 5,545,522, 5,891,636, or 5,716,785). Methods for preparing total and poly(A)⁺ RNA are well known in the art, and are described generally, e.g., in Sambrook, et al., Molecular Cloning: A Laboratory Manual (3rd Edition, 2001). RNA can be extracted from a cell of interest using guanidinium thiocyanate lysis followed by CsCl centrifugation (Chirgwin et al., 1979, Biochemistry 18:5294-5299), a silica gel-based column (e.g., RNeasy (Qiagen, Valencia, Calif.) or StrataPrep (Stratagene, La Jolla, Calif.)), or using phenol and chloroform, as described in Ausubel et al., eds., 1989, Current Protocols In Molecular Biology, Vol. III, Green Publishing Associates, Inc., John Wiley & Sons, Inc., New York, at pp. 13.12.1-13.12.5). Poly(A)⁺ RNA can be selected, e.g., by selection with oligo-dT cellulose or, alternatively, by oligo-dT primed reverse transcription of total cellular RNA. RNA can be fragmented by methods known in the art, e.g., by incubation with ZnCl₂, to generate fragments of RNA.

In one embodiment, total RNA, mRNA, or nucleic acids derived therefrom, are isolated from a sample taken from a bladder cancer patient. Biomarker polynucleotides that are poorly expressed in particular cells may be enriched using normalization techniques (Bonaldo et al., 1996, Genome Res. 6:791-806).

As described above, the biomarker polynucleotides can be detectably labeled at one or more nucleotides. Any method known in the art may be used to label the target polynucleotides. Preferably, this labeling incorporates the label uniformly along the length of the RNA, and more preferably, the labeling is carried out at a high degree of efficiency. For example, polynucleotides can be labeled by oligo-dT primed reverse transcription. Random primers (e.g., 9-mers) can be used in reverse transcription to uniformly incorporate labeled nucleotides over the full length of the polynucleotides. Alternatively, random primers may be used in conjunction with PCR methods or T7 promoter-based in vitro transcription methods in order to amplify polynucleotides.

The detectable label may be a luminescent label. For example, fluorescent labels, bioluminescent labels, chemiluminescent labels, and colorimetric labels may be used in the practice of the invention. Fluorescent labels that can be used include, but are not limited to, fluorescein, a phosphor, a rhodamine, or a polymethine dye derivative. Additionally, commercially available fluorescent labels including, but not limited to, fluorescent phosphoramidites such as FluorePrime (Amersham Pharmacia, Piscataway, N.J.), Fluoredite (Miilipore, Bedford, Mass.), FAM (ABI, Foster City, Calif.), and Cy3 or Cy5 (Amersham Pharmacia, Piscataway, N.J.) can be used. Alternatively, the detectable label can be a radiolabeled nucleotide.

In one embodiment, biomarker polynucleotide molecules from a patient sample are labeled differentially from the corresponding polynucleotide molecules of a reference sample. The reference can comprise polynucleotide molecules from a normal biological sample (i.e., control sample, e.g., urine from a subject not having bladder cancer) or from a bladder cancer reference biological sample, (e.g., urine from a subject having bladder cancer).

Nucleic acid hybridization and wash conditions are chosen so that the target polynucleotide molecules specifically bind or specifically hybridize to the complementary polynucleotide sequences of the array, preferably to a specific array site, wherein its complementary DNA is located. Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self-complementary sequences.

Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook, et al., Molecular Cloning: A Laboratory Manual (3rd Edition, 2001), and in Ausubel et al., Current Protocols In Molecular Biology, vol. 2, Current Protocols Publishing, New York (1994). Typical hybridization conditions for the cDNA microarrays of Schena et al. are hybridization in 5.times.SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Schena et al., Proc. Natl. Acad. Sci. U.S.A. 93:10614 (1993)). Useful hybridization conditions are also provided in, e.g., Tijessen, 1993, Hybridization with Nucleic Acid Probes, Elsevier Science Publishers B.V.; and Kricka, 1992, Nonisotopic Dna Probe Techniques, Academic Press, San Diego, Calif. Particularly preferred hybridization conditions include hybridization at a temperature at or near the mean melting temperature of the probes (e.g., within 51° C., more preferably within 21° C.) in 1 M NaCl, 50 mM MES buffer (pH 6.5), 0.5% sodium sarcosine and 30% formamide.

When fluorescently labeled gene products are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et al., 1996, “A DNA microarray system for analyzing complex DNA samples using two-color fluorescent probe hybridization,” Genome Research 6:639-645, which is incorporated by reference in its entirety for all purposes). Arrays can be scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645 (1996), and in other references cited herein. Alternatively, the fiber-optic bundle described by Ferguson et al., Nature Biotech. 14:1681-1684 (1996), may be used to monitor mRNA abundance levels at a large number of sites simultaneously.

In certain embodiments, the kit comprises a microarray comprising an oligonucleotide that hybridizes to a ROBO1 polynucleotide and an oligonucleotide that hybridizes to a WNT5A polynucleotide. In another embodiment, the microarray further comprises at least one oligonucleotide that hybridizes to at least one reference marker selected from the group consisting of a QRICH1 polynucleotide, a CDC42BPB polynucleotide and a DNMBP polynucleotide. In another embodiment, the microarray further comprises an oligonucleotide that hybridizes to a RARRES1 polynucleotide and an oligonucleotide that hybridizes to a CP polynucleotide. In another embodiment, the microarray further comprises an oligonucleotide that hybridizes to a RARRES1 polynucleotide, an oligonucleotide that hybridizes to a CP polynucleotide, an oligonucleotide that hybridizes to an IGFBP5 polynucleotide, an oligonucleotide that hybridizes to a PLEKHS1 polynucleotide, an oligonucleotide that hybridizes to a BPIFB1 polynucleotide, and an oligonucleotide that hybridizes to a MYBPC1 polynucleotide. In another embodiment, the microarray further comprises an oligonucleotide that hybridizes to a MTRNR2L8 polynucleotide, an oligonucleotide that hybridizes to a VEGFA polynucleotide, and an oligonucleotide that hybridizes to an AKAP12 polynucleotide.

Polynucleotides can also be analyzed by other methods including, but not limited to, northern blotting, nuclease protection assays, RNA fingerprinting, polymerase chain reaction, ligase chain reaction, Qbeta replicase, isothermal amplification method, strand displacement amplification, transcription based amplification systems, nuclease protection (51 nuclease or RNAse protection assays), SAGE as well as methods disclosed in International Publication Nos. WO 88/10315 and WO 89/06700, and International Applications Nos. PCT/US87/00880 and PCT/US89/01025; herein incorporated by reference in their entireties.

A standard Northern blot assay can be used to ascertain an RNA transcript size, identify alternatively spliced RNA transcripts, and the relative amounts of mRNA in a sample, in accordance with conventional Northern hybridization techniques known to those persons of ordinary skill in the art. In Northern blots, RNA samples are first separated by size by electrophoresis in an agarose gel under denaturing conditions. The RNA is then transferred to a membrane, cross-linked, and hybridized with a labeled probe. Nonisotopic or high specific activity radiolabeled probes can be used, including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes. The labeled probe, e.g., a radiolabelled cDNA, either containing the full-length, single stranded DNA or a fragment of that DNA sequence may be at least 20, at least 30, at least 50, or at least 100 consecutive nucleotides in length. The probe can be labeled by any of the many different methods known to those skilled in this art. The labels most commonly employed for these studies are radioactive elements, enzymes, chemicals that fluoresce when exposed to ultraviolet light, and others. A number of fluorescent materials are known and can be utilized as labels. These include, but are not limited to, fluorescein, rhodamine, auramine, Texas Red, AMCA blue and Lucifer Yellow. A particular detecting material is anti-rabbit antibody prepared in goats and conjugated with fluorescein through an isothiocyanate. Proteins can also be labeled with a radioactive element or with an enzyme. The radioactive label can be detected by any of the currently available counting procedures. Isotopes that can be used include, but are not limited to, ³H, ¹⁴C, ³²P, ³⁵S, ³⁶Cl, ³⁵Cr, ⁵⁷Co, ⁵⁸Co, ⁵⁹Fe, ⁹⁰Y, ¹²⁵I, ¹³¹I and ¹⁸⁶Re. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Any enzymes known to one of skill in the art can be utilized. Examples of such enzymes include, but are not limited to, peroxidase, beta-D-galactosidase, urease, glucose oxidase plus peroxidase and alkaline phosphatase. U.S. Pat. Nos. 3,654,090, 3,850,752, and 4,016,043 are referred to by way of example for their disclosure of alternate labeling material and methods.

Nuclease protection assays (including both ribonuclease protection assays and 51 nuclease assays) can be used to detect and quantitate specific mRNAs. In nuclease protection assays, an antisense probe (labeled with, e.g., radiolabeled or nonisotopic) hybridizes in solution to an RNA sample. Following hybridization, single-stranded, unhybridized probe and RNA are degraded by nucleases. An acrylamide gel is used to separate the remaining protected fragments. Typically, solution hybridization is more efficient than membrane-based hybridization, and it can accommodate up to 100 μg of sample RNA, compared with the 20-30 μg maximum of blot hybridizations.

The ribonuclease protection assay, which is the most common type of nuclease protection assay, requires the use of RNA probes. Oligonucleotides and other single-stranded DNA probes can only be used in assays containing 51 nuclease. The single-stranded, antisense probe must typically be completely homologous to target RNA to prevent cleavage of the probe:target hybrid by nuclease.

Serial Analysis Gene Expression (SAGE) can also be used to determine RNA abundances in a cell sample. See, e.g., Velculescu et al., 1995, Science 270:484-7; Carulli, et al., 1998, Journal of Cellular Biochemistry Supplements 30/31:286-96; herein incorporated by reference in their entireties. SAGE analysis does not require a special device for detection, and is one of the preferable analytical methods for simultaneously detecting the expression of a large number of transcription products. First, poly A⁺ RNA is extracted from cells. Next, the RNA is converted into cDNA using a biotinylated oligo (dT) primer, and treated with a four-base recognizing restriction enzyme (Anchoring Enzyme: AE) resulting in AE-treated fragments containing a biotin group at their 3′ terminus. Next, the AE-treated fragments are incubated with streptavidin for binding. The bound cDNA is divided into two fractions, and each fraction is then linked to a different double-stranded oligonucleotide adapter (linker) A or B. These linkers are composed of: (1) a protruding single strand portion having a sequence complementary to the sequence of the protruding portion formed by the action of the anchoring enzyme, (2) a 5′ nucleotide recognizing sequence of the ITS-type restriction enzyme (cleaves at a predetermined location no more than 20 bp away from the recognition site) serving as a tagging enzyme (TE), and (3) an additional sequence of sufficient length for constructing a PCR-specific primer. The linker-linked cDNA is cleaved using the tagging enzyme, and only the linker-linked cDNA sequence portion remains, which is present in the form of a short-strand sequence tag. Next, pools of short-strand sequence tags from the two different types of linkers are linked to each other, followed by PCR amplification using primers specific to linkers A and B. As a result, the amplification product is obtained as a mixture comprising myriad sequences of two adjacent sequence tags (ditags) bound to linkers A and B. The amplification product is treated with the anchoring enzyme, and the free ditag portions are linked into strands in a standard linkage reaction. The amplification product is then cloned. Determination of the clone's nucleotide sequence can be used to obtain a read-out of consecutive ditags of constant length. The presence of mRNA corresponding to each tag can then be identified from the nucleotide sequence of the clone and information on the sequence tags.

Quantitative reverse transcriptase PCR (qRT-PCR) can also be used to determine the expression profiles of biomarkers (see, e.g., U.S. Patent Application Publication No. 2005/0048542A1; herein incorporated by reference in its entirety). The first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TAQMAN PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN RT-PCR can be performed using commercially available equipment, such as, for example, ABI PRISM 7700 sequence detection system. (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700 sequence detection system. The system consists of a thermocycler, laser, charge-coupled device (CCD), camera and computer. The system includes software for running the instrument and for analyzing the data. 5′-Nuclease assay data are initially expressed as Ct, or the threshold cycle. Fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and beta-actin.

A more recent variation of the RT-PCR technique is the real time quantitative PCR, which measures PCR product accumulation through a dual-labeled fluorigenic probe (i.e., TAQMAN probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

Analysis of Biomarker Data

Biomarker data may be analyzed by a variety of methods to identify biomarkers and determine the statistical significance of differences in observed levels of biomarkers between test and reference expression profiles in order to evaluate whether a patient has bladder cancer. In certain embodiments, patient data is analyzed by one or more methods including, but not limited to, multivariate linear discriminant analysis (LDA), receiver operating characteristic (ROC) analysis, principal component analysis (PCA), ensemble data mining methods, significance analysis of microarrays (SAM), cell specific significance analysis of microarrays (csSAM), spanning-tree progression analysis of density-normalized events (SPADE), and multi-dimensional protein identification technology (MUDPIT) analysis. (See, e.g., Hilbe (2009) Logistic Regression Models, Chapman & Hall/CRC Press; McLachlan (2004) Discriminant Analysis and Statistical Pattern Recognition. Wiley Interscience; Zweig et al. (1993) Clin. Chem. 39:561-577; Pepe (2003) The statistical evaluation of medical tests for classification and prediction, New York, N.Y.: Oxford; Sing et al. (2005) Bioinformatics 21:3940-3941; Tusher et al. (2001) Proc. Natl. Acad. Sci. U.S.A. 98:5116-5121; Oza (2006) Ensemble data mining, NASA Ames Research Center, Moffett Field, Calif., USA; English et al. (2009) J. Biomed. Inform. 42(2):287-295; Zhang (2007) Bioinformatics 8: 230; Shen-Orr et al. (2010) Journal of Immunology 184:144-130; Qiu et al. (2011) Nat. Biotechnol. 29(10):886-891; Ru et al. (2006) J. Chromatogr. A. 1111(2):166-174, Jolliffe Principal Component Analysis (Springer Series in Statistics, 2^{nd} edition, Springer, N Y, 2002), Koren et al. (2004) IEEE Trans Vis Comput Graph 10:459-470; herein incorporated by reference in their entireties.)

C. Kits

In yet another aspect, the invention provides kits for diagnosing bladder cancer, wherein the kits can be used to detect the biomarkers of the present invention. For example, the kits can be used to detect any one or more of the biomarkers described herein, which are differentially expressed in samples of a bladder cancer patient and normal subjects (i.e., subjects without bladder cancer). The kit may include one or more agents for detection of biomarkers, a container for holding a biological sample isolated from a human subject suspected of having bladder cancer; and printed instructions for reacting agents with the biological sample or a portion of the biological sample to detect the presence or amount of at least one bladder cancer biomarker in the biological sample. The agents may be packaged in separate containers. The kit may further comprise one or more control reference samples and reagents for performing an immunoassay or microarray analysis.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of diagnosing bladder cancer.

The kits of the invention have a number of applications. For example, the kits can be used to determine if a subject has bladder cancer and to determine if a subject has low-grade or high-grade bladder cancer. In another example, the kits can be used to determine if a patient should be treated for bladder cancer with anti-cancer therapy (e.g., surgery, radiation therapy, chemotherapy, hormonal therapy, immunotherapy, or biologic therapy). In another example, kits can be used to monitor the effectiveness of treatment of a patient having bladder cancer. In a further example, the kits can be used to identify compounds that modulate expression of one or more of the biomarkers in in vitro or in vivo animal models to determine the effects of treatment.

In certain embodiments, the kit comprises agents for measuring the levels of expression of one or more genes selected from Tables 4-10. In another embodiment, the kit further comprises at least one set of PCR primers capable of amplifying a nucleic acid comprising a sequence of a gene selected from Tables 4-10 or its complement. In another embodiment, the kit further comprises at least one probe capable of hybridizing to a nucleic acid comprising a sequence of a gene selected from Table 4-10 or its complement.

In certain embodiments, the kit includes agents for detecting polynucleotides of a biomarker panel comprising a plurality of biomarkers for diagnosing bladder cancer, wherein one or more biomarkers are selected from the group consisting of a WNT5A polynucleotide, a RARRES1 polynucleotide, a ROBO1 polynucleotide, a CP polynucleotide, an IGFBP5 polynucleotide, a PLEKHS1 polynucleotide, a BPIFB1 polynucleotide, and a MYBPC1 polynucleotide.

In certain embodiments, the kit comprises agents for measuring the levels of expression of ROBO1 and WNT5A. In another embodiment, the kit further comprises at least one agent for measuring a level of expression of at least one reference marker selected from the group consisting of QRICH1, CDC42BPB and DNMBP. In another embodiment, the kit further comprises agents for measuring the levels of expression of one or more biomarkers selected from the group consisting of RARRES1, CP, IGFBP5, PLEKHS1, BPIFB1, and MYBPC1. In another embodiment, the kit comprises agents for measuring the levels of expression of RARRES1 and CP. In another embodiment, the kit further comprises agents for measuring the levels of expression of one or more additional genes selected from Tables 4-10.

In another embodiment, the kit comprises agents for measuring the levels of expression of one or more genes selected from the group consisting of MTRNR2L8, VEGFA, and AKAP12. In another embodiment, the kit comprises agents for measuring the levels of expression of MTRNR2L8, VEGFA, and AKAP12.

In another embodiment, the kit further comprises at least one set of PCR primers capable of amplifying a nucleic acid comprising a sequence of a gene selected from Table 5 or Table 6, or its complement.

In another embodiment, the kit further comprises at least one probe capable of hybridizing to a nucleic acid comprising a sequence of a gene selected from Table 5 or Table 6 or its complement.

In certain embodiments, the kit comprises a microarray comprising an oligonucleotide that hybridizes to a ROBO1 polynucleotide and an oligonucleotide that hybridizes to a WNT5A polynucleotide. In another embodiment, the microarray further comprises an oligonucleotide that hybridizes to a CDC42BPB polynucleotide.

In another embodiment, the microarray further comprises an oligonucleotide that hybridizes to a RARRES1 polynucleotide and an oligonucleotide that hybridizes to a CP polynucleotide.

In another embodiment, the microarray further comprises an oligonucleotide that hybridizes to a RARRES1 polynucleotide, an oligonucleotide that hybridizes to a CP polynucleotide, an oligonucleotide that hybridizes to an IGFBP5 polynucleotide, an oligonucleotide that hybridizes to a PLEKHS1 polynucleotide, an oligonucleotide that hybridizes to a BPIFB1 polynucleotide, and an oligonucleotide that hybridizes to a MYBPC1 polynucleotide.

In another embodiment, the microarray further comprises an oligonucleotide that hybridizes to a MTRNR2L8 polynucleotide, an oligonucleotide that hybridizes to a VEGFA polynucleotide, and an oligonucleotide that hybridizes to an AKAP12 polynucleotide.

### III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Deep Sequencing of Urinary RNAs for Development of Bladder Cancer Molecular Diagnostics

Introduction

We applied RNA-seq as a discovery tool to identify a panel of bladder cancer-specific urinary mRNA markers. Sequencing RNA extracted directly from urine sediment from bladder cancer patients and controls resulted in an average of 100 million sequencing reads per sample. Genes selected based on the RNA-seq analysis were evaluated using quantitative real-time polymerase chain reaction (qPCR) in a training cohort. This data was used to select a 3-marker panel consisting of two cancer-specific genes (ROBO1, WNT5A) and one reference gene (CDC42BPB). The diagnostic accuracy of the 3-marker panel was evaluated in an independent patient cohort and was compared to urine cytology.

Methods

Study Design

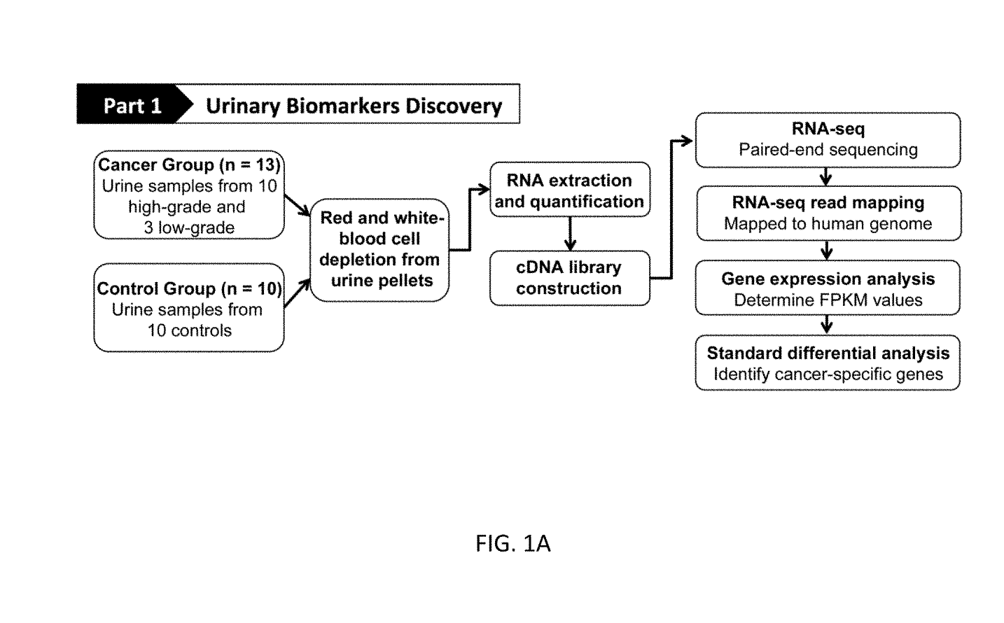

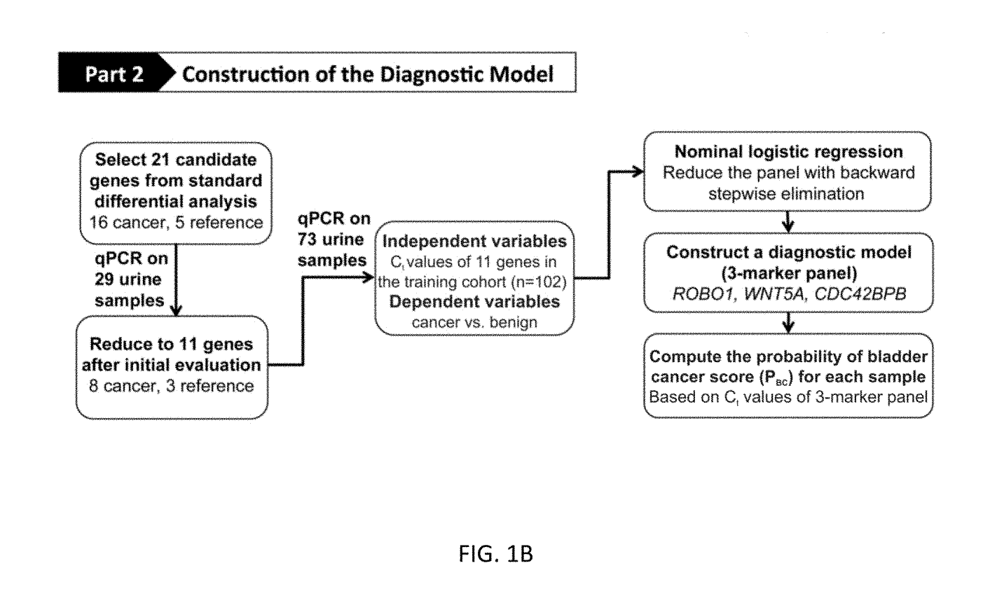

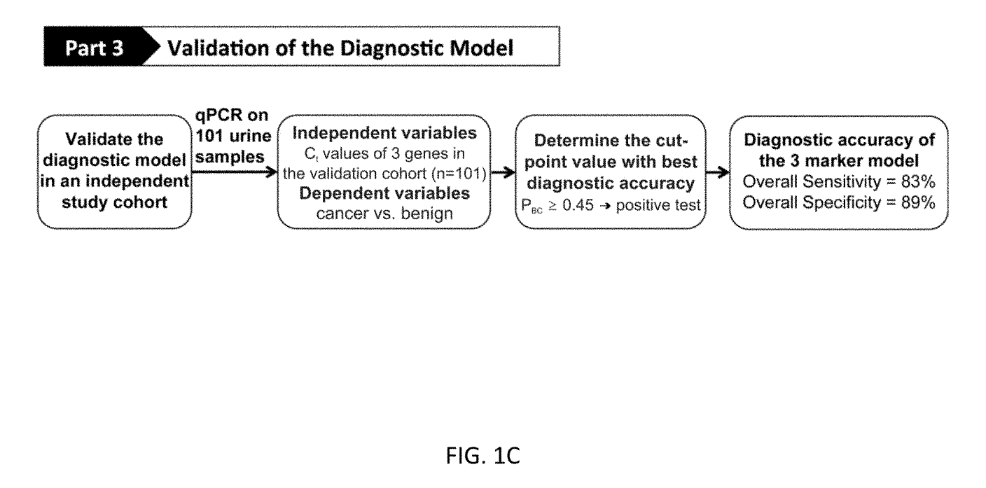

The study protocol was approved by the Stanford University Institutional Review Board and Veterans Affairs Palo Alto Health Care System (VAPAHCS) Research and Development Committee. All patients were recruited from VAPAHCS. The study was divided into 3 parts: 1) biomarker discovery, 2) construction of the diagnostic model, and 3) validation of the diagnostic model (FIG. 1). For each part, urine samples were collected from bladder cancer and control subjects. Patients of both genders≥18 years old were eligible for enrollment. Patients with other malignant urological disease were excluded. For biomarker discovery, urine samples were collected from 23 subjects (13 bladder cancer and 10 controls) for RNA-seq analysis. To construct the diagnostic model, expression of candidate genes identified by RNA-seq was analyzed in urinary RNA extracts from a training cohort of 102 urines samples (50 bladder cancer and 52 controls) using qPCR. The 3-marker diagnostic panel was then validated in 101 urine samples (47 bladder cancer and 54 controls) to determine assay diagnostic sensitivity and specificity. Urine cytology was performed on a subset of samples per routine clinical care.

Patient Population and Samples

“Bladder cancer-evaluation” group are patients with no prior history of bladder cancer and undergoing urological work-up, primarily for hematuria. “Bladder cancer-surveillance” group are patients with prior history of bladder cancer undergoing routine surveillance. “Control” group are patients with non-neoplastic urological diseases or healthy volunteers≥35 years old. Urine samples were categorized as cancer or benign based on corresponding tissue histopathology from TUR or cystoscopic biopsy when available. For urine samples without a matching tissue sample from bladder cancer evaluation or surveillance patients, diagnosis was based on cystoscopic findings. Urine samples from patients with non-neoplastic urological diseases (e.g. kidney stones) and healthy control groups that did not undergo cystoscopy were presumed negative for bladder cancer based on clinical history. Cytology results were considered positive when reported as suspicious or malignant and negative when reported as atypical or negative.

Urine Sample Preparation and RNA Extraction for RNA-Seq

For RNA-seq, urine samples (10-750 ml) were processed within two hours of collection. Urine sediment was collected by centrifugation for 15 minutes at 500×g and pellets were washed 3 times with PBS. Washed urine sediment was depleted of red and white blood cells (RBCs and WBCs). RBCs were selectively lysed by addition of 1000 μl of 10-fold diluted RBC lysis solution (Miltenyi Biotec). Remaining cells were collected by centrifugation at 300×g for 5 minutes and cell pellets washed 3 times with PBS. To deplete WBCs, cells were incubated for 15 minutes at 4° C. with 80 μl of magnetic-activated cell sorting (MACS) buffer (PBS, 0.5% bovine serum albumin, and 2 mM EDTA) and 20 μl of anti-CD45 magnetic micro-beads. Then 1 ml of MACS buffer was added and cells collected by centrifuged at 300×g for 15 minutes at 4° C. The cells were re-suspended in 500 μl MACS buffer and applied to a MACS LD column (Miltenyi Biotec). The column was washed twice with 1 ml MACS buffer and the total effluent collected. For RNA extraction, urothelial cells were collected by centrifugation and resuspended in 1 ml TRIzol (Invitrogen) and stored at −80° C. Total RNA from the urothelial cells was extracted with TRIzol reagent followed by DNA degradation with RQ1 RNase-free DNase (Promega) then purification on RNeasy MinElute Cleanup columns (Qiagen) according to the manufacturer's instructions. An Agilent 2100 Bioanalyzer and RNA Pico chips were used for total RNA quantification and qualification analysis. RNA concentration and RNA integrity number (RIN) were determined for each sample.

Library Preparation and RNA-Seq

The cDNAs were synthesized from samples with a total RNA 6 ng in 12 μl of nuclease-free water using the Ovation RNA Seq System V2 kit (NuGEN Technologies) according to manufacturer's instructions. cDNAs were fragmented with S-Series Focused-ultrasonicator (Covaris). To enrich for cDNAs>300 bases in length, cDNAs were size fractionated by incubating with 0.8 volume of Agencourt AMPure XP beads (Beckman Coulter) for 10 minutes followed by bead separation on 96S super magnet plate (Alpaque) for 10 minutes. Beads were then washed three times with 80% ethanol and air-dried for 15 minutes on the magnetic plate. cDNA products were eluted with 102 μl of RNase-free water and quantity was measured by spectrophotometry (NanoDrop). Barcoded sequencing libraries were prepared using a NEBNext Ultra DNA Library Prep Kit for Illumina (New England Biolabs) and cDNA libraries were enriched with the Agencourt AMPure XP beads (Beckman Coulter) as described above and eluted with 30 μl of buffer EB (Qiagen). Sequencing libraries were paired-end sequenced with reads of 100 bases long on the Illumina HiSeq 2000 at Stanford Stem Cell Institute Genome Center.

RNA-Seq Gene Expression Analysis and Candidate Selection

RNA-seq reads were mapped to the human genome (GRCh38) using TopHat. Mapped reads were assembled and gene expression analysis performed using Cufflinks software tools. The sequence fragments were normalized to take into account both gene length and mapped reads for each sample, to measure the relative abundance of genes based on fragments per kilobase of exon per million fragments mapped (FPKM). Standard differential analysis based on the FPKM values was performed to compare gene expression profiles of control, bladder cancer, HG, and LG using Cuffdiff software to identify and prioritize cancer-specific genes by the fold-change of genes with a false discovery rate (q-value)≤0.05. To select against candidate markers also highly expressed in blood cells, the gene expression profiles of potential candidate genes was examined in blood cells using gene expression commons, an open platform for absolute gene expression profiling in the human hematopoietic system (Seita et al. (2012) PLoS One 7(7):e40321).

qPCR Gene Expression Analysis

For qPCR analysis, urine sediments were collected and RNA extracted, purified and quantitated as described above, but without blood cell depletion. cDNAs for all samples were generated using the Ovation RNA Seq System V2 kit (NuGEN Technologies) according to manufacturer's instructions, and in 4 samples (1 LG, 1 HG, and 2 controls in the training cohort), cDNA synthesis was also carried out with a High-Capacity RNA-cDNA kit (Applied Biosystems) for comparison. cDNAs were enriched for >300 base fragments with the AXYPREPMAG PCR Clean-up bead solution (AXYPREP) and bead separation on 96S super magnet plate (Alpaque), eluted and quantitated as described above for RNA-seq analysis. The cDNA products were amplified in single reactions using TAQMAN Gene Expression Assays (Applied Biosystems). The TAQMAN primers and probes were selected to span an exon-exon junction without detecting genomic DNA (Tables 8 and 9). The qPCR reactions were performed in triplicate. For each reaction, 10 ng cDNA in 9 μl was mixed with 10 μl 2× TAQMAN Gene Expression Master Mix (Applied Biosystems) and 1 μl 20×TAQMAN Gene Expression Assay solution in a final volume of 20 μl and amplified in an ABI PRISM 7900 HT sequence detection system (Applied Biosystems). Reactions were heated to 50° C. for 2 minutes and 95° C. for 10 minutes before being cycled 40 times at 95° C. for 15 seconds and 60° C. for 1 minute. The qPCR results were processed with SDS 2.4 and RQ manager software packages (Applied Biosystems). An automated threshold and baseline were used to determine the cycle threshold value (Cₜ). The mean of the triplicate measurements of Cₜ was used for data analysis. For genes with undetermined Cₜ values, Cₜ value of 45 was assigned. Samples with Cₜ 37 for 2 of 3 reference genes (QRICH1, CDC42BPB, and DNMBP) in the training cohort and the 1 reference gene (CDC42BPB) in the validation cohort were excluded from analysis due to insufficient RNA quantity or quality.

Statistical Analysis

For initial diagnostic model construction, 21 markers were tested with 29 urine samples. The relative expression level of cancer genes was evaluated as the geometric average of the Cₜ of 5 reference genes—Cₜ of the cancer gene (ΔCₜ). The initial panel was narrowed to 11 markers (8 cancer and 3 reference) for testing of an additional 73 urine samples. The Cₜ values of the 11-marker panel were used for statistical analysis with JMP Pro 12 (SAS Institute Inc.). Univariate logistic regression was used to study the predictive ability of the 11 markers on the cancer status with the odds ratios (ORs) with 95% confidence intervals (CIs), area under the curve (AUC), and p-value. Multiple logistic regression with backward stepwise elimination using stopping rule of entering p-value=0.25 and leaving p-value=0.05 was performed to reduce the panel of markers. A reference marker was included in the model as a sample adequacy control and to normalize cell numbers. Cₜ values of 3-marker signature (ROBO1, WNT5A, and CDC42BPB) were used for calculating the probability of bladder cancer score (P_{BC}) of each sample: P_{BC}=exp [A]/(1+exp [A]) with A=19.82-0.43×ROBO1 Cₜ−0.56×WNT5A Cₜ+0.33×CDC42BPB Cₜ. Receiver operating characteristic (ROC) curve and AUC for the 3-marker panel were generated and calculated with the JMP Pro 12 software. Empirical ROC curve for the cytology report was estimated from ordinal empirical data with 4 categories (negative, atypical, suspicious, and malignant) (16). Sensitivity and specificity for each category was determined and the ROC curve was generated with 4 sets of data point connected by straight line. AUC of the ROC curve was calculated using R software.

Results

Study Participants

Between 2013 and 2016, 186 human subjects were recruited and 226 urine samples collected and processed. Subject demographic and clinicopathologic characteristics are shown in Table 1. Urine samples were collected from 1) patients undergoing bladder cancer evaluation (BC-evaluation) who presented with hematuria (n=78), suspicious urine cytology (n=2) or suspicious mass in computer tomography (n=3); 2) patients with known history of bladder cancer undergoing surveillance cystoscopy (BC-surveillance, n=118); 3) patients with non-neoplastic urological diseases including benign prostatic hyperplasia (n=2), urolithiasis (n=2), urinary tract infections (n=1) and indwelling ureteral stents (n=3) (other non-neoplastic urological diseases); and 4) healthy male volunteers age>35 years with no prior history of cancer or active urological issues (healthy controls, n=17).

Urinary Biomarkers Discovery

To identify candidate urinary biomarkers, RNA-seq was applied to 10 urine samples from patients with HG bladder cancer, 3 samples from patients with LG bladder cancer, and 10 control samples (Table 2). To reduce non-urothelial cell sequences related to the blood-cell-associated transcriptome, RBCs and WBCs were depleted prior to total RNA isolation for sequencing. Notably, more RNA was extracted from cancer samples than from controls with a mean total RNA concentration per urine volume of 0.98 ng/ml for cancer and 0.080 ng/ml for controls, likely due to a higher concentration of urothelial cells in urine of cancer patients. As shown in Table 2, 41-313 million paired-reads were generated per sample and 13-72.5% of the reads could be mapped to human genome. Two control samples had a low percentage of mapped reads, sample 4 with 13% and sample 9 with 27%, suggestive of sample contamination and were excluded from further analysis. Standard differential analysis based on FPKM values was performed for pairwise comparison of the gene expression profiles of control, HG, LG and combined HG and LG bladder cancer. Comparison of control and combined bladder cancer identified 418 differentially expressed genes, 281 over-expressed and 137 under-expressed in bladder cancer. Comparison of control and HG samples yielded 105 differentially express genes, 74 over-expressed and 31 under-expressed in HG. Comparison of control and LG samples identified, 17 differentially express genes, 8 over-expressed and 9 under-expressed in LG. When comparing LG to HG samples, 3 genes were over-expressed in HG. The full panel of differentially expressed genes, prioritized by fold change of FPKM value is listed in Tables 4-7.

Biomarker Selection Based on RNA-Seq

To select for candidate biomarkers, genes known to be highly expressed in blood cells were excluded from further validation to minimize false positive signals due to hematuria and inflammation (Seita et al., supra). Candidate bladder cancer-specific genes were chosen from the control vs. HG and the control vs. combined bladder cancer comparisons. Fifteen of the candidate genes selected (CP, PLEKHS1, MYBPC1, ROBO1, RARRES1, WNT5A, AKR1C2, AR, IGFBP5, ENTPD5, SLC14A1, FBLN1, SYBU, STEAP2, and GPD1L) were overexpressed in HG samples with fold-change above control ranging from 3.10 to 7.39. One bladder cancer specific gene, BPIFB1, identified in control vs. combined bladder cancer comparison had a 6.65-fold increase in cancer. All of the candidate cancer specific genes were recognized among the top 30 genes in the control vs. bladder cancer comparison. The cuffdiff output for the 16 bladder cancer-specific genes selected for the validation in the training study cohort is shown in Table 8. In order to find a suitable reference gene to control urinary RNA quantity, 5 genes (QRICH1, CDC42BPB, USP39, ITSN1, and DNMBP) with uniform expression level, mean FPKM value ˜4, and standard deviation (SD)≤0.25 among all 23 RNA-seq samples were selected for investigation (Table 9).

Biomarker Validation in the Training Cohort

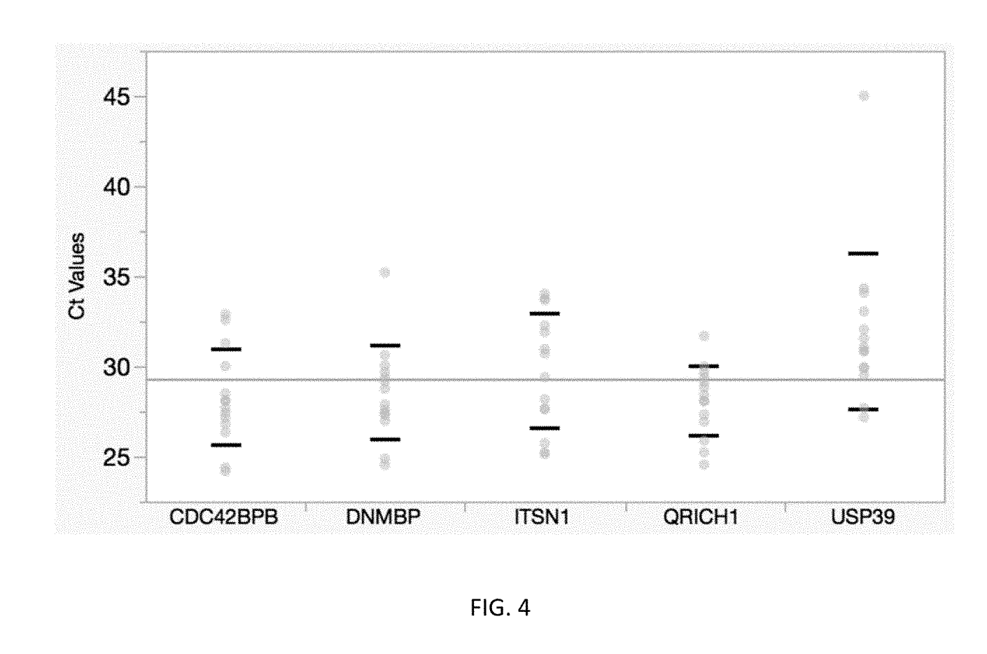

Candidate biomarkers were validated in a training cohort of cancer and control urine samples to confirm expression level and select a panel with best diagnostic performance for bladder cancer. Gene expression of an initial panel of 16 cancer-specific and 5 reference genes was determined by qPCR in 29 urine samples (16 cancer and 15 controls). Uniform expression of the candidate reference genes was evaluated and the qPCR Ct values from control and cancer samples were collected and compiled (FIG. 4). Among the candidates references genes QRICH1, CDC42BPB and DNMBP had the most similar Ct values (˜28) and least variability (SD range 2.0 to 2.6), indicating they are stably expressed and suitable for data normalization in qPCR experiments. Based on the relative expression of the cancer genes normalized to the reference genes (ΔCt), 8 of the cancer genes (WNT5A, RARRES1, ROBO1, CP, IGFBP5, PLEKHS1, BPIFB1, and MYBPC1) were selected for additional testing. These 8 cancer and 3 reference genes were evaluated in an additional 73 urine samples (34 cancer and 39 controls).

To confirm that qPCR validation results were not biased by the reverse transcriptase method used to generate cDNA from urinary RNA, qPCR experiments with the 11 candidate genes were run on 4 samples (2 bladder cancer, and 2 controls) with cDNAs produced using two different kits (NuGEN Technologies and Applied Biosystems). After the qPCR data were normalized using the geometric average of the 3 reference genes, the relative expression levels of the 8 cancer genes was consistent between methods (data not shown) suggesting reverse transcriptase kit did not introduce bias in the gene expression analysis.

Construction of the Diagnostic Model

Univariate logistic analysis of Cₜ values of the 11 candidate genes in training cohort urine samples was performed to evaluate predictive accuracy for bladder cancer for each candidate. The 8 bladder cancer markers were all significant predictors (p-value<0.0001). WNT5A, RARRES1, ROBO1 and CP were the strongest predictors of bladder cancer with odds ratios ranging from 1.65 to 2.12 and AUCs≥0.9 (Table 10). Although the reference markers were chosen as sample adequacy and reference levels for the number of cells in the sample, two of the reference markers, CDC42BPB (p=0.0476) and DNMBP (p<0.0001), were significant predictors of bladder cancer, likely due to higher concentration of urothelial cells in bladder cancer samples.

Multiple logistic regression analysis of Cₜ values of the 11 candidate genes in the training cohort was used to construct a diagnostic model equation. ROBO1, WNT5A and CDC42BPB were identified as having relevant, non-redundant diagnostic values for constructing an equation to calculate a probability of bladder cancer score (P_{BC}):

P_{BC}=exp[A]/1+exp[A]

A=19.82-0.43×ROBO1Cₜ−0.56×WNT5A Cₜ+0.33×CDC42BPB Cₜ

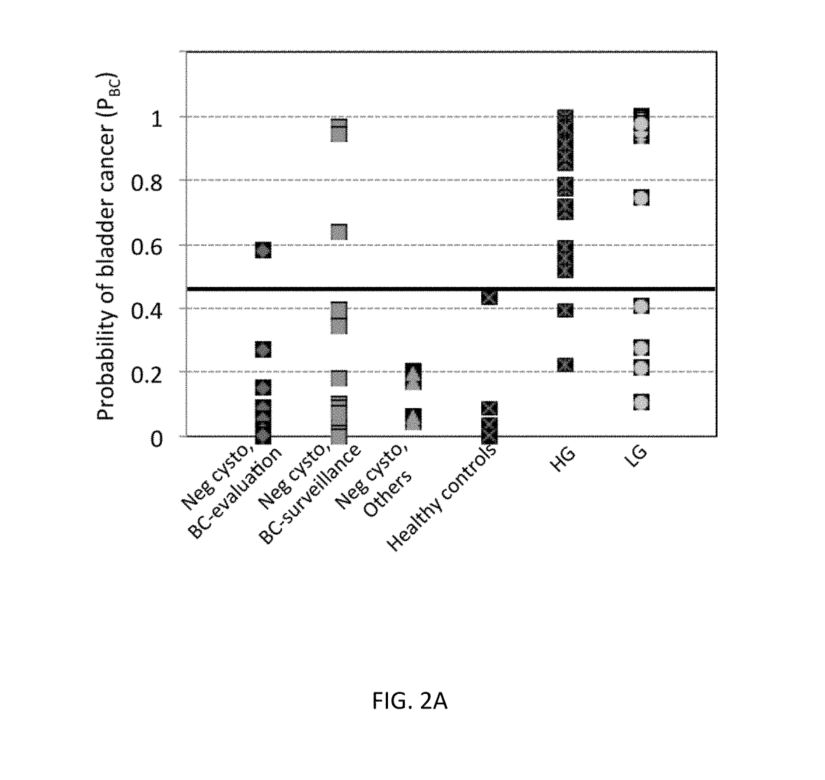

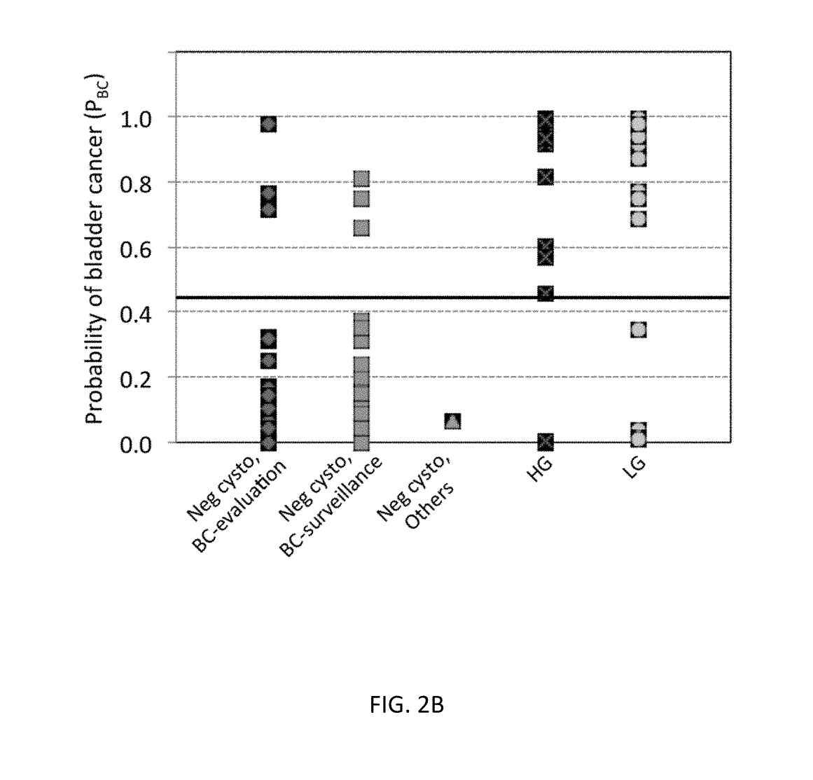

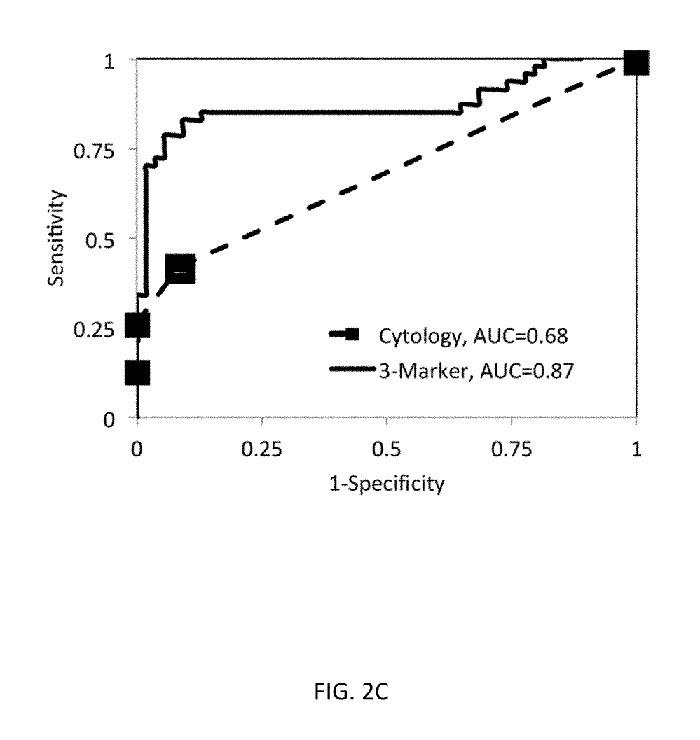

Using this equation, the P_{BC} for each sample in the training cohort was calculated (FIG. 2A). A P_{BC}≥0.45-cutoff was designated a positive test as it gave the best overall combination of sensitivity and specificity at 88% and 92% respectively (Table 3). In 81 samples, the diagnostic accuracy of the 3-marker panel using P_{BC}≥0.45 cutoff was compared to cytology. While the overall specificity of the 3-marker panel was modestly lower than cytology, the overall sensitivity was much better, 88% for the 3-marker panel compared to 19% for cytology.

Validation of the Diagnostic Model

The 3-marker panel of ROBO1, WNT5A, and CDC42BPB was evaluated by qPCR in an independent validation set of 101 urine samples (47 cancer and 54 controls) from 86 patients (Table 1, FIG. 2B). Using P_{BC}≥0.45 as the threshold for positive test, the overall sensitivity and specificity for the 3-marker panel was 83% and 89%, respectively (Table 3). The diagnostic performance of 3-marker panel was also compared with cytology on a subset of samples (n=89) with an AUC of 0.87, which was significantly more accurate than the diagnosis by cytology with an AUC of 0.68 (p<0.01) (FIG. 2C). As in the training cohort, sensitivity of the 3-marker panel was higher than cytology but specificity was lower.

Using the 3-Marker Panel for Bladder Cancer Surveillance

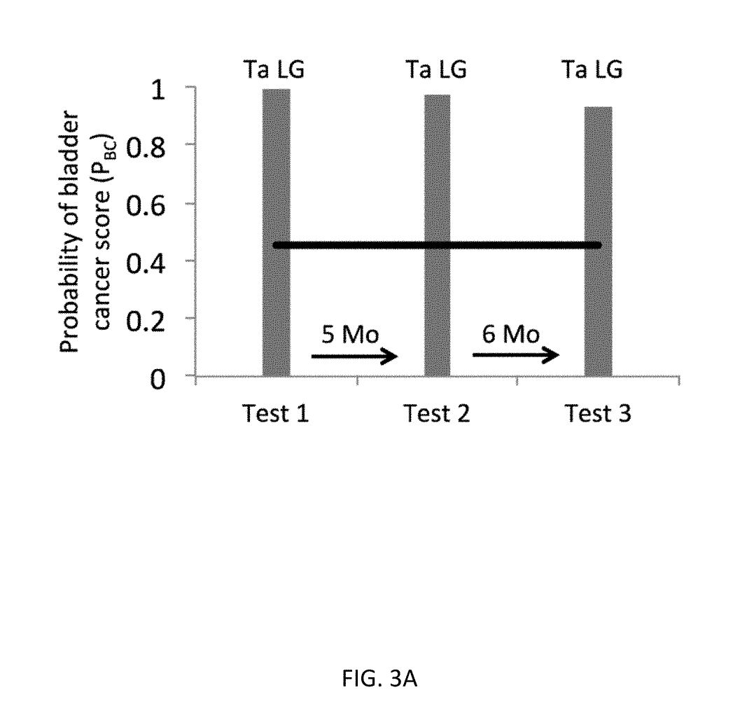

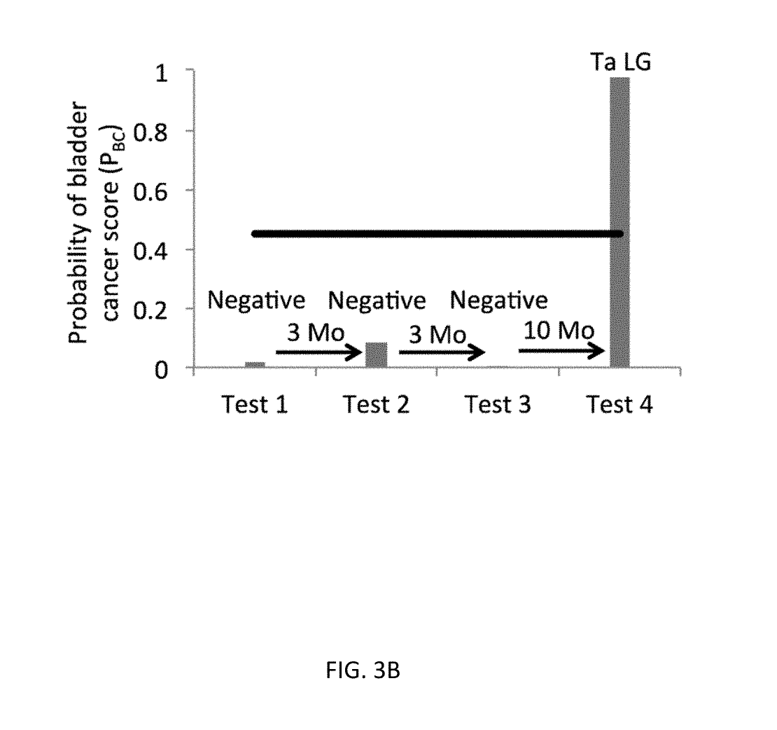

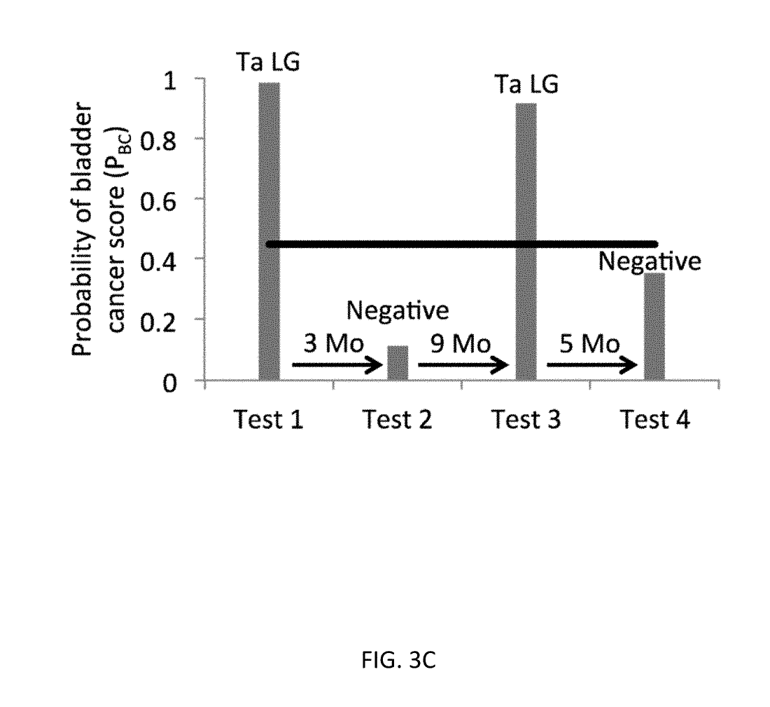

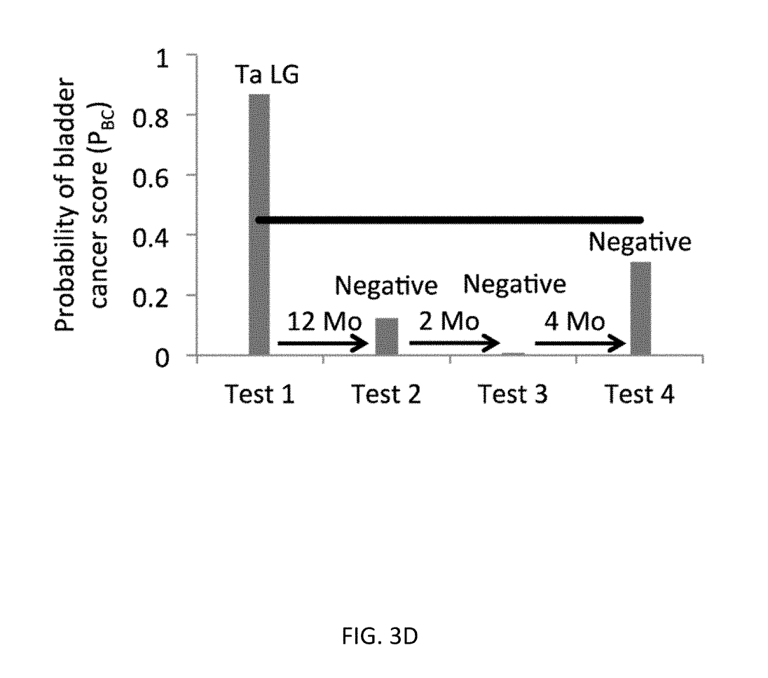

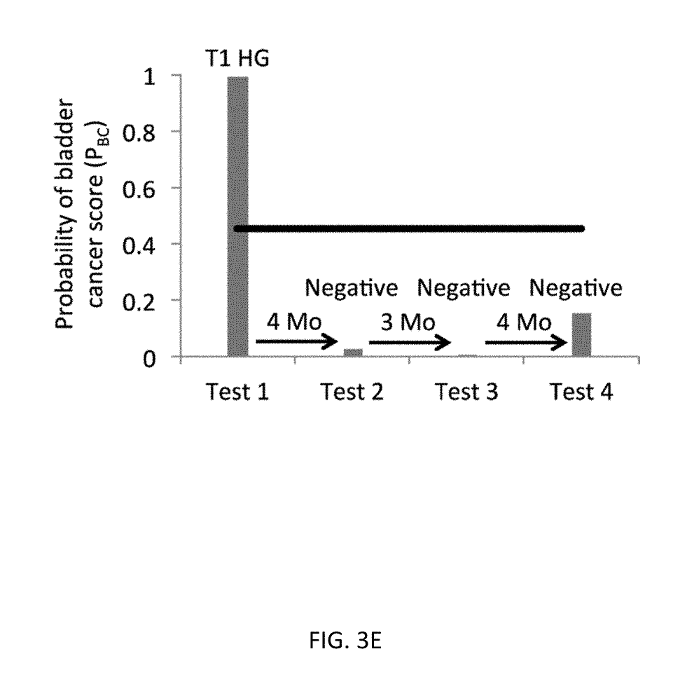

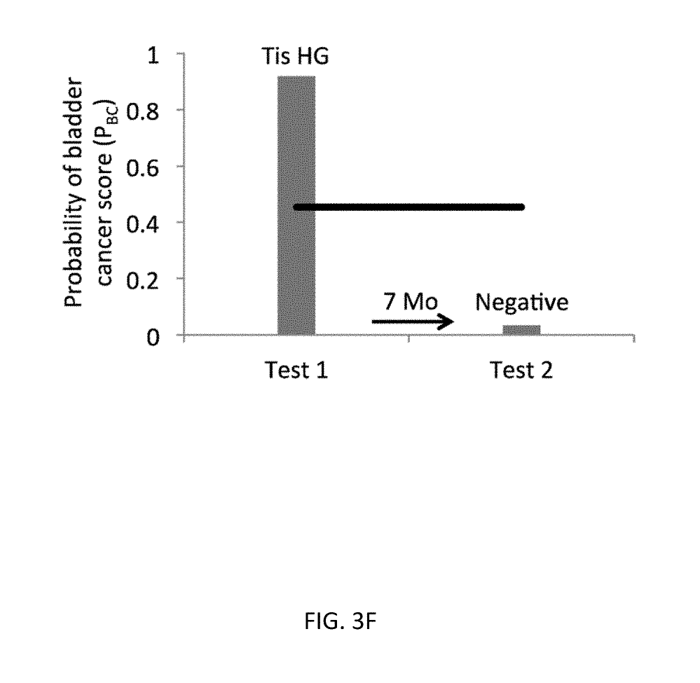

To explore the potential of using the 3-marker panel urine test for bladder cancer surveillance, we evaluated its test performance in serially collected urine samples from six patients. For each patient, 2 to 4 urine samples were collected over 7 to 18 months. The results from the 3-marker panel were compared with cystoscopic and/or pathologic findings. In all patients, the 3-marker panel was concordant with cystoscopic and/or pathologic results, both in cancer positive and negative scenarios (FIG. 3).

In a patient with LG Ta bladder cancer (FIG. 3A), the 3-marker panel was positive at the initial diagnosis and two subsequent cancer recurrences, whereas cytology remained negative throughout, indicating that the 3-marker panel is a better adjunct to cystoscopy for this patient. In another patient with prior history of LG with focal HG bladder cancer, the patient had 3 negative cystoscopy and 3 matched negative 3-marker urine tests (FIG. 3B). At the time of tissue-confirmed recurrence 16 months later, the 3-marker panel also turned positive. The concordance of the 3-panel marker with cystoscopy suggest that the use of the panel may reduce the frequency of cystoscopic surveillance in selected patients. Similar findings are seen in two other patients with Ta LG cancer (FIGS. 3C-3D), in which the 3-marker panel paralleled negative cystoscopies and biopsy-proven recurrences.

In patients with HG T1 (FIG. 3E) and TIS (FIG. 3F) at the time of study entry, both cytology and the 3-marker panel were positive at cancer diagnosis and negative during surveillance. Notably, the patient in FIG. 3F underwent induction BCG following the diagnosis of TIS. The surveillance cystoscopy following BCG identified an erythematous patch on the anterior bladder wall. The appropriately negative 3-marker panel (FIG. 3F, test 2) suggests that, at least in this case, the test remained reliable after BCG and did not falsely identify inflammation as bladder cancer.

Discussion

While most bladder cancers are non-muscle invasive at initial diagnosis, the high recurrence rate and potential to progress to invasive disease necessitates frequent surveillance cystoscopy, contributing to bladder cancer as one of the most expensive cancers to treat (Yeung et al. (2014) PharmacoEconomics 32(11):1093-1104). To date, a non-invasive test with sufficient accuracy to reduce the frequency of cystoscopy in low-risk patients, while providing timely treatment in high-risk patients, has remained elusive. For development of a urine-base bladder cancer test, we reasoned that direct analysis of exfoliated urothelial cells, rather than tissue biopsies, would yield higher translational potential for biomarker discovery. We applied RNA-seq for unbiased gene expression analysis of urinary cells and demonstrated the success of extracting high quality RNA and generating high quality sequencing for identifying a new 3-marker panel (ROBO1, WNT5A and CDC42BPB) for molecular diagnosis of bladder cancer.

Identification of differentially expressed genes between cancer and benign tissues is a common starting point for biomarker discovery. Development of next generation sequencing technologies that allow for high sensitivity, resolution, throughput and speed have advanced research on biomarker discovery for cancer diagnosis, assessing prognosis, and directing treatment monitoring (Shyr et al. (2013) Biol. Proced. Online 15(1):4; Zhang et al. (2013) Cancer Letters 340(2):149-150; Petric et al. (2015) Clujul Med. 88(3):278-287; Yli-Hietanen et al. (2015) Chin. J. Cancer 34(10):423-426). RNA-seq has emerged as a powerful tool for unbiased interrogation of gene expression as well as identification of splice variants and non-coding RNAs (Wang et al. (2009) Nature Reviews Genetics 10(1):57-63). Direct application of RNA-seq to urine has been limited, given the relatively low cellularity and heterogeneity of urine samples that may impact RNA integrity. To address these issues, we processed the entire volume urine sample within 2 hours of collection to maximize the number of cells and preserve RNA integrity. To enrich the urothelial cell fraction in the urine sediment and reduce transcripts related to the blood cells, RBCs and WBCs were depleted from the samples. Additionally, candidate genes identified by RNA-seq that are also known to be highly expressed in blood cells were excluded from marker validation. Using this approach, we enriched target cells and genes specific to bladder cancer while reducing confounding markers of inflammation that are abundant in setting of urinary tract infection and post-intravesical BCG administration.

Supporting the validity of our discovery approach, several of the bladder cancer specific genes identified through RNA-seq have been implicated as biomarkers in bladder and other cancers. CP, which has the highest fold increase in cancer compared to control in our screen (Table 4), encodes a feroxidase enzyme and was previously identified in a proteomic screen as a urinary biomarker of bladder cancer (Chen et al. (2012) J. Proteomics 75(12):3529-3545) and as a serum biomarker in other cancers (Turecky et al. (1984) Klin. Wochenschr 62(4):187-189). IGFBP5, another top candidate gene, was previously found to be upregulated in bladder cancer by tissue microarray analysis and is part of the Cxbladder 5-marker panel for bladder cancer diagnosis described below (O'Sullivan et al. (2012) J. Urol. 188(3):741-747; Holyoake et al. (2008) Clin. Cancer Res. 14(3):742-749). The two cancer specific genes in our 3-marker panel were also previously implicated in tumor formation and progression. ROBO1 is a promoter of tumor angiogenesis and overexpressed in both human bladder cancer tissue and cultured cell lines (Li et al. (2015) Int. J. Clin. Exp. Pathol. 8(9):9932-9940; Legg et al. (2008) Angiogenesis 11(1):13-21). WNT5A is a secreted glycoprotein that plays an important regulatory role in embryogenesis, including regulation of cell polarity and migration. WNT5A expression decreases after development and upregulation in adult tissue has been implicated in oncogenesis (Kumawat et al. (2016) Cell Mol. Life Sci. 73(3):567-587). In bladder cancer, WNT5A protein expression correlated positively with the histological grade and pathological stage (Malgor et al. (2013) Diagnostic Pathology 8:139; Endo et al. (2015) Int. Rev. Cell Mol. Biol. 314:117-48).

Several urine tests have been approved for clinical use in bladder cancer. However, due to inadequate sensitivity (particularly in LG cancer) and specificity in inflammatory conditions, current guidelines on NMIBC do not recommend their routine use for surveillance or initial work-up (Chang et al. (2016) J Urol. 196(4):1021-1029; Babjuk et al. (2013) European Urology 64(4):639-653). Fluorescence in situ hybridization (UroVysion) and immunocytochemistry (ImmunoCyt) incorporate molecular markers with microscopic evaluation of urine cells with overall better sensitivity but lower specificity than conventional cytology (Urquidi et al. (2016) Oncotarget 7(25):38731-38740; Chou et al. (2015) Ann. Intern. Med. 163(12):922-931). Protein biomarker assays nuclear matrix protein 22 (NMP22) and bladder tumor antigen (BTA) offer the potential for simple, objective tests (Vrooman et al. (2008) European Urology 53(5):909-916). Both tests have higher sensitivity but lower specificity than cytology, especially in patients with inflammation and infection in the urinary tract (Todenhofer et al. (2012) Urology 79(3):620-624; van Rhijn et all. (2005) European Urology 47(6):736-748).

Recent efforts to improve urine-based diagnostics for bladder cancer have focused on multiplex detection of mRNAs that are differentially expressed between cancer and non-cancerous tissues. A general strategy uses microarray analysis of bladder cancer tissue samples for target selection, followed by validation in urine samples. One panel, Cxbladder (Pacific Edge, Dunedin, New Zealand), assays urinary expression of bladder cancer markers CDC2, HOXA13, MDK and IGFBP5, as well as inflammation biomarker CXCR2 to reduce false positive tests (Holyoake et al. (2008) Clin. Cancer Res. 14(3):742-749). In a multicenter prospective study of 485 patients presenting with gross hematuria, the Cxbladder assay had an overall sensitivity of 81% (97% for HG, 69% for LG) and specificity of 85% (O'Sullivan et al. (2012) J. Urol. 188(3):741-747). Another assay under development by BiofinaDX (Madrid, Spain) uses a 2, 5, 10 or 12 gene signature for urinary detection of bladder cancer (Ribal et al. (2016) Eur. J. Cancer 54:131-138). The 12-gene signature was first identified by microarray analysis of bladder cancer tumor tissue then validated in urine samples (Mengual et al. (2010) Clin. Cancer Res. 16(9):2624-2633). In a multicenter prospective study of 525 samples, the 12-marker panel was narrowed to two (IGF2 and MAGEA) with an overall sensitivity of 81% (89% for HG, 68% for LG) and specificity of 91% (Mengual et al. (2014) J. Urol. 191(1):261-269; Ribal et al. (2016) Eur. J. Cancer 54:131-138).

Improving the diagnostic sensitivity of LG is one of the central goals of urine-based diagnostics, as the majority of bladder cancer patients present with LG disease. Our diagnostic model consisting of ROBO1, WNT5A, and CDC42BPB, had an overall sensitivity of 83% and specificity of 89%. Compared to Cxbladder and BiofinaDX, our overall sensitivity was similar, and subset analysis showed improved sensitivity for LG cancer (83% vs. 69% and 68%) (O'Sullivan et al., supra; Ribal et al., supra). The improved sensitivity may be due in part to our urine-based biomarker discovery strategy to target mRNA that are not only differentially expressed in bladder cancer but also maintain stability in urine. Additionally, concentrating the cellular fraction from the entire urine sample may account for superior detection of LG tumors that shed fewer cells.

One strength of our study is the serial testing for a cohort of patients over their course of bladder cancer surveillance (FIG. 3). The consistent results between cystoscopy and the 3-marker panel suggest that the test is a dependable adjunct for cancer surveillance. This may be especially true in the setting of an initial positive 3-marker urine test indicating that the markers are upregulated in the tumor. Based on our dataset, we set the threshold for a positive test at P_{BC}≥0.45 in both bladder cancer evaluation and surveillance populations. In the clinical scenario of using the urine test to prescreen patients before cystoscopy, sensitivity may be considered more important than specificity as the clinical outcome of missing cancer is worse than negative cystoscopy. To maximize the sensitivity, the threshold for a positive test may be set lower for surveillance than in evaluation populations as recurrent bladder tumors tend to be smaller than primary tumors (Chang et al., supra), which may result in a lower cancer P_{BC} value. For example, using a lower cutoff for bladder cancer surveillance than evaluation was found to improve sensitivity of the NMP22 test (Boman et al. (2002) J. Urol. 167(1):80-83). Other efforts that may improve the accuracy of bladder cancer diagnostics include integration of the urine tests with the clinical characteristic (Lotan et al. (2014) J. Urol. 192(5):1343-1348; Ajili et al. (2013) Ultrastruct. Pathol. 37(3):191-195; Lotan et al. (2009) BJU International 103(10):1368-1374). For example, Kavalieris el al. developed an integrated model consisting of both Cxbladder gene expression urine test and patient characteristic variables such as gender, age, smoking history and frequency of gross hematuria for use to triage patients for hematuria workup but with a low probability of bladder cancer (Kavalieris et al. (2015) BMC Urology 15:23).

As this is a case-control study with a relatively small sample size, a large, prospective, multicenter study is required to further evaluate the 3-marker panel and potentially refine population specific P_{BC} thresholds. It will also be valuable to evaluate the 3-marker panel in patients undergoing BCG where the performance of urine cytology is poor due to an increase of inflammatory cells in urine (Chang et al., supra; Lopez-Beltran et al. (2002) J. Clin. Pathol. 55(9):641-647). Our approach of selecting against markers of inflammation suggests our 3-marker may be useful for assessing patient response to BCG treatment. Further, as subjects were selected retrospectively, valid bladder cancer prevalence estimates cannot be obtained. A larger prospective study will allow us to calculate negative and positive predictive values of the test and set a P_{BC} cutoff to maximize the negative predictive value, which may be useful for reducing the need for cystoscopy. With a larger sample size, we can also assess whether supplementing our gene expression model with a phenotypic model of risk stratification provides an improved resource for clinical decisions, particularly for patients with scores near the P_{BC} threshold (Lotan et al. (2010) Urol. Oncol-Semin. Ori. 28(4):441-448). Lastly, further interrogation of our RNA-seq dataset may yield insights into bladder cancer biology, identify rare splice variants and other RNA targets (e.g. miRNA, lncRNA) that were enriched through our sample preparation strategy.

### CONCLUSIONS

Using RNA-seq as a discovery tool, we have demonstrated the feasibility of obtaining high quality sequencing data from urine sediments for RNA expression profiling. Through qPCR evaluation and linear logistic analysis, we generated an equation to predict bladder cancer probability based on the urinary expression of ROBO1, WNT5A and CDC42BPB. The overall sensitivity for both the high-grade and low-grade samples was superior to urine cytology. A prospective multicenter clinical study should be conducted to further validate the 3 marker signature for detection, surveillance, and post-BCG populations.

TABLE 1
Demographic and clinicopathogic features of the study cohorts.

| | Biomarker | | | | | |
|---|---|---|---|---|---|---|
| | Discovery | | Diagnostic Model | | Validation | |
| Demographic | Benign | Cancer | Benign | Cancer | Benign | Cancer |
| features | (n = 10) | (n = 13) | (n = 52) | (n = 50) | (n = 54) | (n = 47) |
| Average age (range)^{a} | >35 | 72.8 | 67.3 | 71.8 | 70.8 | 71.4 |
| | | (58-90) | (30-89) | (53-93) | (29-100) | (55-91) |
| Gender: male/ | 10/0 | 13/0 | 52/0 | 50/0 | 53/1 | 47/0 |
| female, n | | | | | | |
| BC-evaluation | — | 8 | 15 | 23 | 22 | 15 |
| BC-surveillance | — | 5 | 23 | 27 | 31 | 32 |
| Healthy/other | 10 | — | 14 | — | 1 | — |
| controls | | | | | | |

| Clinicopathologic | | | | |
|---|---|---|---|---|
| features^{b} | | Cancer (n = 13) | Cancer (n = 50) | Cancer (n = 47) |
| Grade | Low | | 19 | 29 |
| | High | 10 | 31 | 18 |
| Clinical | Papillary | | | |
| Stage | Ta | 8 | 28 | 36 |
| | T1 | 1 | 10 | 4 |
| | ≥ T2 | 2 | 3 | 5 |
| | Papillary + CIS | | | |
| | Ta | — | 1 | 2 |
| | T1 | 1 | — | — |
| | T2 | 1 | 2 | — |
| | CIS | — | 6 | — |

Abbreviation:
CIS, carcinoma in situ.
^{a}Average age and range does not include healthy controls as specific ages were not collected for this group.
^{b}Clinicopathologic features are available only for bladder cancer patients.

TABLE 2
Summary of urine samples used for RNA-seq transcriptome
profiling.

| Sample | Clinicopathologic | Urine | Total RNA | RIN | | % of mapped |
|---|---|---|---|---|---|---|
| Number | features | volume (ml) | concentration (ng) | (1-10)^{a} | Number of reads | reads |
| 1 | Control | 200 | 7.1 | 9.4 | 88,922,624 | 35.3 |
| 2 | Control | 75 | 13.6 | 5.3 | 84,926,624 | 37.8 |
| 3 | Control | 190 | 6.1 | 9.2 | 82,152,466 | 58.3 |
| 4 | Control | 150 | 11.6 | 2.5 | 202,122,232 | 13.0 |
| 5 | Control | 200 | 11.1 | 6.6 | 211,454,432 | 47.6 |
| 6 | Control | 435 | 51.0 | 5.6 | 261,575,308 | 48.4 |
| 7 | Control | 327 | 27.5 | 2.7 | 313,362,582 | 44.3 |
| 8 | Control | 750 | 17.4 | 3.7 | 59,641,782 | 54.8 |
| 9 | Control | 460 | 13.3 | 4.9 | 54,411,982 | 27.0 |
| 10 | Control | 500 | 81.7 | 6.6 | 73,908,808 | 52.9 |
| 11 | Ta HG | 50 | 9.7 | 9.5 | 77,299,864 | 35.2 |
| 12 | Ta HG | 176 | 57.4 | 7.6 | 100,212,450 | 72.5 |
| 13 | Ta HG | 140 | 8.8 | 3.1 | 76,097,546 | 54.1 |
| 14 | Ta HG | 125 | 28.0 | 2.7 | 90,664,046 | 59.5 |
| 15 | Ta HG | 82 | 72.8 | 3.8 | 57,041,308 | 59.8 |
| 16 | T1 HG | 110 | 128.8 | 7.0 | 41,764,318 | 64.6 |
| 17 | T2 HG | 60 | 63.7 | 6.7 | 95,286,642 | 70.1 |
| 18 | T2 HG | 115 | 110.2 | 8.6 | 67,061,502 | 68.1 |
| 19 | T1 HG + CIS | 125 | 101.4 | 6.9 | 91,298,356 | 39.0 |
| 20 | T2 HG + CIS | 133 | 603.8 | 6.2 | 70,401,502 | 65.9 |
| 21 | Ta LG | 80 | 79.4 | 7.7 | 58,109,042 | 65.3 |
| 22 | Ta LG | 215 | 110.0 | 6.3 | 55,461,696 | 46.8 |
| 23 | Ta LG | 68 | 67.9 | 6.2 | 48,072,074 | 61.8 |

Abbreviations:
CIS, carcinoma in situ.
^{a}The RNA integrity number (RIN) is an algorithm for evaluating the integrity of RNA with a value of 1 to 10, with 10 being the least degraded.

TABLE 3
Summary of diagnostic performance for bladder cancer prediction on urine based on
the 3-marker panel using ROBO1, WNT5A, and CDC42BPB and cytology in both training and validation
cohorts with the cutoff of P_{BC} ≥ 0.45 giving a positive test.

| | | Training Cohort | | Validation Cohort | |
|---|---|---|---|---|---|
| | | 3-Marker Panel | Cytology^{a} | 3-Marker Panel | Cytology^{a} |
| Sensitivity | All Cancer | 88% (44/50) | 19% (8/42) | 83% (39/47) | 25% (10/40) |
| | HG | 94% (29/31) | 30% (7/23) | 83% (15/18) | 50% (7/14) |
| | LG | 79% (15/19) | 5% (1/19) | 83% (24/29) | 12% (3/26) |
| Specificity | All Non-Cancer | 92% (48/52) | 97% (38/39) | 89% (48/54) | 100% (49/49) |
| | Negative BC evaluation | 93% (14/15) | 100% (15/15) | 86% (19/22) | 100% (19/19) |
| | Negative BC surveillance | 87% (20/23) | 96% (22/23) | 90% (28/31) | 100% (30/30) |
| | Healthy/Other Controls | 100% (14/14) | 100% (1/1) | 100% (1/1) | N/A |

^{a}Cytology reports were only available for a subset of samples.

TABLE 4
Differentially expressed genes identified in comparison
of cancer to control based on urinary RNA-seq.

| Gene | Log2 fold change^{a} | q-value^{b} |
|---|---|---|
| CP | 7.51 | 0.00564 |
| BPIFB1 | 6.65 | 0.03433 |
| MYBPC1 | 5.73 | 0.00564 |
| PTPRZ1 | 5.67 | 0.01837 |
| PLEKHS1 | 5.66 | 0.00564 |
| LOC440895 | 5.22 | 0.04314 |
| PDE8B | 4.91 | 0.01210 |
| ROBO1 | 4.85 | 0.00564 |
| SCGB2A1 | 4.82 | 0.03053 |
| CHP2 | 4.72 | 0.03484 |
| WNT5A | 4.72 | 0.00564 |
| CFTR | 4.67 | 0.00564 |
| RARRES1 | 4.39 | 0.00564 |
| IGFBP5 | 4.31 | 0.00564 |
| SLC14A1 | 3.97 | 0.00564 |
| AR | 3.96 | 0.00564 |
| ENTPD5 | 3.92 | 0.00564 |
| SYBU | 3.87 | 0.00564 |
| STEAP2 | 3.84 | 0.00564 |
| IL20RA | 3.81 | 0.03001 |
| AKR1C2 | 3.79 | 0.00886 |
| MYB | 3.65 | 0.00564 |
| GPD1L | 3.33 | 0.00564 |
| CLIC6 | 3.30 | 0.00564 |
| TMEM98 | 3.29 | 0.00564 |
| EEF2K | 3.27 | 0.00564 |
| MPPED2 | 3.20 | 0.00886 |
| CAPN13 | 3.20 | 0.00564 |
| SIDT1 | 3.17 | 0.00564 |
| FBLN1 | 3.09 | 0.01643 |
| TNFSF15 | 3.06 | 0.00564 |
| PEX11A | 3.05 | 0.00564 |
| MBOAT1 | 3.04 | 0.00886 |
| SRGAP3 | 3.04 | 0.00564 |
| SPTSSB | 3.04 | 0.00564 |
| TP63 | 3.03 | 0.03717 |
| PBX1 | 3.01 | 0.00564 |
| MUC15 | 2.96 | 0.00564 |
| HECW2 | 2.96 | 0.04949 |
| GNPTAB | 2.94 | 0.00564 |
| ENPP5 | 2.93 | 0.00564 |
| TTLL7 | 2.91 | 0.00564 |
| BMP3 | 2.86 | 0.00564 |
| PPM1L | 2.79 | 0.00564 |
| MGST1 | 2.78 | 0.00564 |
| VIPR1 | 2.77 | 0.00886 |
| AGR2 | 2.75 | 0.00886 |
| LOC92249 | 2.73 | 0.00886 |
| ALDH5A1 | 2.72 | 0.00564 |
| TLR3 | 2.71 | 0.00564 |
| TSPAN12 | 2.71 | 0.00564 |
| ERLIN1 | 2.68 | 0.00564 |
| PDK3 | 2.66 | 0.00564 |
| ATP2A3 | 2.63 | 0.00564 |
| SLC12A2 | 2.60 | 0.02193 |
| CCSER1 | 2.60 | 0.02193 |
| ZNF436 | 2.59 | 0.01837 |
| PPP1R12B | 2.59 | 0.02313 |
| HNMT | 2.59 | 0.03717 |
| MEIS2 | 2.57 | 0.02444 |
| HMGCS2 | 2.56 | 0.00564 |
| FXYD3 | 2.53 | 0.03053 |
| NFIA | 2.51 | 0.01459 |
| ZNF704 | 2.51 | 0.00564 |
| PCDH7 | 2.49 | 0.04146 |
| HERC2P9 | 2.48 | 0.00886 |
| PLCE1 | 2.47 | 0.01210 |
| LPAR5 | 2.47 | 0.01210 |
| CAT | 2.44 | 0.02721 |
| CDC42BPA | 2.42 | 0.00564 |
| CCDC169 | 2.42 | 0.04701 |
| SDR42E1 | 2.41 | 0.00564 |
| GSDMB | 2.41 | 0.03885 |
| AUTS2 | 2.40 | 0.02031 |
| BANK1 | 2.39 | 0.02550 |
| SYT2 | 2.39 | 0.01643 |
| RGMB | 2.36 | 0.00886 |
| ATP8A1 | 2.36 | 0.01837 |
| IDH1 | 2.35 | 0.00564 |
| PPIL3 | 2.35 | 0.03951 |
| MANSC1 | 2.34 | 0.02444 |
| C1orf21 | 2.34 | 0.01459 |
| FAM162A | 2.34 | 0.00886 |
| NBEA | 2.31 | 0.02444 |
| CASP6 | 2.30 | 0.03602 |
| CYB561 | 2.30 | 0.02600 |
| CRABP2 | 2.29 | 0.01459 |
| NHS | 2.29 | 0.02031 |
| TARBP1 | 2.29 | 0.01210 |
| FUT10 | 2.29 | 0.01459 |
| PRKAB2 | 2.28 | 0.00564 |
| WDR52 | 2.28 | 0.00564 |
| SLC25A12 | 2.28 | 0.02600 |
| PTGFRN | 2.26 | 0.00564 |
| ACSL5 | 2.25 | 0.00886 |
| C22orf29 | 2.24 | 0.03001 |
| PRKAR2B | 2.24 | 0.01837 |
| AHCYL2 | 2.22 | 0.03484 |
| XBP1 | 2.22 | 0.00564 |
| ARHGAP35 | 2.20 | 0.00564 |
| ERP27 | 2.20 | 0.01210 |
| DIS3L | 2.19 | 0.00886 |
| TRIT1 | 2.18 | 0.01837 |
| RNF128 | 2.18 | 0.03053 |
| CAMK2D | 2.18 | 0.03602 |
| TCN1 | 2.17 | 0.00564 |
| RAB27B | 2.16 | 0.00886 |
| FUT8 | 2.16 | 0.01459 |
| GGT6 | 2.16 | 0.02550 |
| PPARG | 2.14 | 0.03307 |
| HSD17B11 | 2.14 | 0.01210 |
| ERMP1 | 2.13 | 0.00564 |
| CTDSPL | 2.13 | 0.03001 |
| TLE1 | 2.11 | 0.01210 |
| TBX3 | 2.10 | 0.02444 |
| ENAH | 2.10 | 0.04439 |
| FBXO9 | 2.10 | 0.02444 |
| ENTPD3 | 2.09 | 0.01210 |
| ST6GALNAC1 | 2.09 | 0.04635 |
| RAPGEFL1 | 2.09 | 0.04314 |
| TSHZ1 | 2.08 | 0.03828 |
| FAM210B | 2.07 | 0.02600 |
| MLEC | 2.07 | 0.03366 |
| NUDT9 | 2.07 | 0.01837 |
| EPAS1 | 2.06 | 0.00564 |
| TBC1D30 | 2.06 | 0.02313 |
| NUDT4 | 2.05 | 0.02444 |
| LYPD6B | 2.04 | 0.03053 |
| TIMM21 | 2.04 | 0.00886 |
| ZNF514 | 2.04 | 0.02444 |
| ZC3H8 | 2.03 | 0.03053 |
| FAM83H- | 2.03 | 0.03484 |
| PIAS3 | 2.03 | 0.03484 |
| ZNF439 | 2.02 | 0.01459 |
| ARV1 | 2.02 | 0.04314 |
| POF1B | 2.02 | 0.01643 |
| ERBB2 | 2.02 | 0.04146 |
| SCCPDH | 2.00 | 0.04146 |
| NSMCE4A | 2.00 | 0.01837 |
| TMEM242 | 1.99 | 0.04439 |
| BTBD3 | 1.99 | 0.04213 |
| SLC39A6 | 1.99 | 0.02600 |
| ZNF280D | 1.99 | 0.03433 |
| USP46 | 1.99 | 0.03756 |
| PDCL3 | 1.99 | 0.04579 |
| NAALADL2 | 1.98 | 0.04146 |
| PREP | 1.98 | 0.01643 |
| AKAP1 | 1.98 | 0.02313 |
| SRPRB | 1.97 | 0.02600 |
| BBS9 | 1.97 | 0.02550 |
| PDCD4 | 1.97 | 0.02846 |
| TCEAL4 | 1.97 | 0.02600 |
| CYP4F12 | 1.96 | 0.03885 |
| ALAD | 1.95 | 0.02313 |
| THOC1 | 1.95 | 0.02193 |
| FAM174B | 1.95 | 0.04146 |
| C2orf43 | 1.93 | 0.03484 |
| ZNF605 | 1.93 | 0.04213 |
| MTPAP | 1.92 | 0.03001 |
| ZNF507 | 1.92 | 0.03951 |
| SRI | 1.91 | 0.01837 |
| SPTLC3 | 1.91 | 0.03433 |
| TMEM168 | 1.91 | 0.04213 |
| MTA3 | 1.90 | 0.03183 |
| PIGU | 1.90 | 0.02193 |
| PLCH1 | 1.89 | 0.02600 |
| ZNHIT6 | 1.87 | 0.03756 |
| GPR89A | 1.87 | 0.03484 |
| DIMT1 | 1.87 | 0.04122 |
| ZZZ3 | 1.87 | 0.03366 |
| DHCR24 | 1.87 | 0.03756 |
| LONP2 | 1.87 | 0.00564 |
| IQCB1 | 1.87 | 0.02031 |
| TPD52 | 1.86 | 0.02313 |
| F5 | 1.86 | 0.03828 |
| CFH | 1.85 | 0.02846 |
| TMEM260 | 1.84 | 0.04213 |
| MRFAP1L1 | 1.84 | 0.00564 |
| ZNF558 | 1.83 | 0.03053 |
| AMOT | 1.82 | 0.02600 |
| SPICE1 | 1.81 | 0.00886 |
| LTV1 | 1.81 | 0.03484 |
| SLC37A3 | 1.81 | 0.04439 |
| PTCD3 | 1.79 | 0.00564 |
| GOLGA8B | 1.79 | 0.04439 |
| KLHDC2 | 1.79 | 0.03951 |
| GOLGA8A | 1.79 | 0.00564 |
| ZNF318 | 1.79 | 0.04401 |
| TTC37 | 1.78 | 0.00564 |
| ZFP90 | 1.76 | 0.04439 |
| ADD3 | 1.76 | 0.04038 |
| ITGB1BP1 | 1.76 | 0.03756 |
| TTC21B | 1.76 | 0.00564 |
| DROSHA | 1.74 | 0.02846 |
| SLC25A20 | 1.73 | 0.04949 |
| HADH | 1.72 | 0.00564 |
| RHOU | 1.72 | 0.01643 |
| CYB5A | 1.71 | 0.04913 |
| SRPX2 | 1.71 | 0.04439 |
| URI1 | 1.70 | 0.02721 |
| INSIG2 | 1.69 | 0.01643 |
| LPCAT3 | 1.68 | 0.00564 |
| RAB3GAP1 | 1.68 | 0.00886 |
| KIAA1244 | 1.67 | 0.00886 |
| MTIF2 | 1.66 | 0.01643 |
| DENND2D | 1.64 | 0.02193 |
| CCDC14 | 1.63 | 0.00564 |
| OARD1 | 1.62 | 0.00564 |
| HGSNAT | 1.62 | 0.00564 |
| DYRK2 | 1.61 | 0.02444 |
| GORASP2 | 1.61 | 0.01643 |
| TBL2 | 1.60 | 0.04522 |
| CAB39L | 1.60 | 0.03951 |
| MAVS | 1.59 | 0.04439 |
| UTP20 | 1.58 | 0.04719 |
| KDELR2 | 1.57 | 0.01643 |
| RYK | 1.57 | 0.03484 |
| ZMYM4 | 1.56 | 0.01459 |
| ZCCHC7 | 1.56 | 0.04635 |
| IARS2 | 1.55 | 0.01643 |
| NRIP1 | 1.55 | 0.00564 |
| PIGN | 1.55 | 0.03756 |
| MAGED1 | 1.54 | 0.03307 |
| NBPF14 | 1.54 | 0.02193 |
| HERC2P2 | 1.53 | 0.01459 |
| NBPF10 | 1.51 | 0.00886 |
| THOC2 | 1.50 | 0.02846 |
| METAP1 | 1.50 | 0.01837 |
| CARD6 | 1.49 | 0.01643 |
| ACACA | 1.48 | 0.04522 |
| LTN1 | 1.46 | 0.02031 |
| NBPF20 | 1.46 | 0.02721 |
| YLPM1 | 1.44 | 0.02031 |
| NEO1 | 1.44 | 0.03885 |
| TMEM245 | 1.43 | 0.01837 |
| STT3A | 1.43 | 0.02031 |
| FAM20B | 1.43 | 0.02193 |
| ATR | 1.43 | 0.02313 |
| STT3B | 1.42 | 0.02313 |
| TMEM181 | 1.42 | 0.02550 |
| EEA1 | 1.41 | 0.02550 |
| MCCC2 | 1.40 | 0.02600 |
| MIA3 | 1.40 | 0.02550 |
| ANKRD27 | 1.40 | 0.02313 |
| CHD6 | 1.40 | 0.03366 |
| SEC63 | 1.40 | 0.03885 |
| STRBP | 1.40 | 0.03756 |
| CPSF3 | 1.39 | 0.01643 |
| RARS | 1.38 | 0.03433 |
| EIF3E | 1.38 | 0.02550 |
| EPT1 | 1.38 | 0.03433 |
| OCRL | 1.38 | 0.04146 |
| KIAA0319L | 1.37 | 0.03307 |
| PIK3R1 | 1.36 | 0.02721 |
| UBR3 | 1.36 | 0.03951 |
| MKL2 | 1.35 | 0.03307 |
| MBTPS1 | 1.35 | 0.03053 |
| SCAPER | 1.34 | 0.04038 |
| EIF3M | 1.33 | 0.03366 |
| ARL1 | 1.33 | 0.04122 |
| TOPBP1 | 1.33 | 0.04522 |
| MTMR4 | 1.33 | 0.04401 |
| SSR1 | 1.33 | 0.02444 |
| IKBKAP | 1.32 | 0.03756 |
| TMEM39A | 1.32 | 0.03828 |
| MIOS | 1.32 | 0.04038 |
| RPRD1A | 1.32 | 0.03828 |
| ZFYVE20 | 1.30 | 0.03433 |
| PCYOX1 | 1.29 | 0.03756 |
| NUP107 | 1.29 | 0.04579 |
| NAA25 | 1.29 | 0.04913 |
| MED1 | 1.28 | 0.03885 |
| OPHN1 | 1.27 | 0.04213 |
| EPB41L4A | 1.27 | 0.04635 |
| FBXW2 | 1.27 | 0.04635 |
| PAPSS1 | 1.27 | 0.03433 |
| GFPT1 | 1.26 | 0.04579 |
| IMPAD1 | 1.24 | 0.04949 |
| TIMMDC1 | 1.24 | 0.04701 |
| KLHL12 | 1.24 | 0.04949 |
| MPP7 | 1.21 | 0.04949 |
| JOSD1 | −1.23 | 0.04949 |
| ANKLE2 | −1.31 | 0.03183 |
| IFIT2 | −1.43 | 0.03885 |
| ATP6V1B2 | −1.56 | 0.03366 |
| TNFRSF10D | −1.57 | 0.04719 |
| EGR1 | −1.65 | 0.04146 |
| SERPINB1 | −1.65 | 0.04401 |
| CDCP1 | −1.68 | 0.04146 |
| VASP | −1.68 | 0.03366 |
| ACAT1 | −1.70 | 0.04719 |
| KIAA0247 | −1.70 | 0.04122 |
| ABCA12 | −1.73 | 0.03828 |
| R3HDM4 | −1.75 | 0.03053 |
| MYO9B | −1.76 | 0.03366 |
| TMCC3 | −1.78 | 0.02846 |
| PADI1 | −1.78 | 0.03433 |
| GCH1 | −1.80 | 0.03885 |
| DNTTIP1 | −1.81 | 0.04122 |
| SHB | −1.81 | 0.03717 |
| SH3BGRL3 | −1.82 | 0.02600 |
| BHLHE40 | −1.82 | 0.04146 |
| HIST1H2BC | −1.83 | 0.04522 |
| SGTA | −1.84 | 0.03366 |
| PFKP | −1.85 | 0.04439 |
| PAF1 | −1.86 | 0.04701 |
| NABP1 | −1.88 | 0.04579 |
| NDRG2 | −1.89 | 0.02846 |
| SLC25A37 | −1.90 | 0.03433 |
| GTPBP1 | −1.91 | 0.03053 |
| C15orf39 | −1.93 | 0.04213 |
| MED16 | −1.93 | 0.02721 |
| XDH | −1.95 | 0.01459 |
| HS3ST1 | −1.95 | 0.02550 |
| ARRB2 | −1.95 | 0.03602 |
| TALDO1 | −1.96 | 0.02550 |
| TOM1 | −1.97 | 0.03717 |
| TUBA1A | −1.99 | 0.02600 |
| CENPBD1P1 | −1.99 | 0.03484 |
| SCNN1B | −2.02 | 0.02721 |
| RHOG | −2.02 | 0.00564 |
| PADI2 | −2.03 | 0.00564 |
| IL1RN | −2.04 | 0.04038 |
| DUSP6 | −2.06 | 0.03756 |
| HIST1H1C | −2.07 | 0.03183 |
| CLTB | −2.09 | 0.03756 |
| MAP1LC3B2 | −2.10 | 0.00564 |
| KRT15 | −2.13 | 0.02444 |
| GNB2 | −2.13 | 0.00886 |
| FRMD8 | −2.14 | 0.01643 |
| LAMB3 | −2.17 | 0.02313 |
| CREM | −2.18 | 0.00564 |
| PVR | −2.19 | 0.02313 |
| MAP2K3 | −2.19 | 0.02193 |
| FAM129B | −2.19 | 0.03183 |
| PDZK1IP1 | −2.22 | 0.02600 |
| CPPED1 | −2.22 | 0.04146 |
| HCAR2 | −2.23 | 0.01643 |
| PIM3 | −2.25 | 0.02193 |
| MYADM | −2.26 | 0.03053 |
| SLC16A3 | −2.26 | 0.03183 |
| CYFIP2 | −2.37 | 0.04038 |
| PLEKHH2 | −2.38 | 0.02600 |
| MIDN | −2.39 | 0.00564 |
| ECM1 | −2.39 | 0.03828 |
| RAPGEF1 | −2.40 | 0.00886 |
| PFKFB3 | −2.45 | 0.02031 |
| EHD1 | −2.47 | 0.00564 |
| GK | −2.49 | 0.00564 |
| PMEPA1 | −2.49 | 0.04817 |
| SOD2 | −2.53 | 0.03053 |
| CXCL6 | −2.54 | 0.02721 |
| NPNT | −2.56 | 0.04719 |
| CDKN1A | −2.57 | 0.03307 |
| GLS | −2.59 | 0.02600 |
| SOCS3 | −2.64 | 0.00886 |
| LRG1 | −2.64 | 0.00564 |
| LEMD1 | −2.68 | 0.03951 |
| DENND3 | −2.71 | 0.00564 |
| THBS1 | −2.72 | 0.00564 |
| LRRK2 | −2.73 | 0.00564 |
| UPP1 | −2.79 | 0.02721 |
| GADD45B | −2.80 | 0.00564 |
| CSRNP1 | −2.90 | 0.00564 |
| TNFAIP3 | −2.92 | 0.01210 |
| ABLIM2 | −2.92 | 0.03366 |
| SIRPA | −2.93 | 0.03366 |
| GNA15 | −2.93 | 0.00564 |
| MAL | −3.00 | 0.00564 |
| DCDC2 | −3.00 | 0.00886 |
| KDM6B | −3.04 | 0.00564 |
| CHST15 | −3.04 | 0.00564 |
| C14orf105 | −3.17 | 0.02313 |
| KCTD11 | −3.20 | 0.00564 |
| LAMC2 | −3.24 | 0.01459 |
| TYMP | −3.29 | 0.03828 |
| RALGDS | −3.38 | 0.02550 |
| SERPINA1 | −3.43 | 0.00564 |
| IL4I1 | −3.48 | 0.04635 |
| ICAM1 | −3.51 | 0.00564 |
| EGR2 | −3.52 | 0.04719 |
| THEMIS2 | −3.56 | 0.02031 |
| NREP | −3.62 | 0.01210 |
| FCGR2A | −3.65 | 0.01643 |
| RBP1 | −3.70 | 0.04579 |
| PLAUR | −3.79 | 0.01459 |
| PAX8 | −4.08 | 0.02031 |
| IL1R2 | −4.09 | 0.04122 |
| NCF2 | −4.16 | 0.04401 |
| NR4A1 | −4.17 | 0.00564 |
| FCGR3A | −4.17 | 0.03053 |
| ARHGAP25 | −4.24 | 0.03602 |
| APBB1IP | −4.40 | 0.03484 |
| SPP1 | −4.42 | 0.00886 |
| MRVI1-AS1 | −4.43 | 0.02193 |
| EGR3 | −4.46 | 0.04701 |
| TAGAP | −4.47 | 0.04719 |
| CYTIP | −4.55 | 0.04635 |
| TREML2 | −4.82 | 0.04719 |
| C5AR1 | −4.88 | 0.04579 |
| IKZF1 | −4.98 | 0.03951 |
| CCL2 | −5.00 | 0.01210 |
| GPR65 | −5.02 | 0.02846 |
| FXYD2 | −5.12 | 0.04949 |
| FCER1G | −5.18 | 0.04719 |
| PTGS1 | −5.29 | 0.03885 |
| PTPRC | −5.41 | 0.00886 |
| SLC11A1 | −5.43 | 0.04817 |
| MT2A | −5.50 | 0.00564 |
| MT1M | −5.77 | 0.02550 |
| ZEB2 | −5.86 | 0.01837 |
| MT1A | −6.01 | 0.01459 |
| CD14 | −6.04 | 0.01459 |
| CCDC85B | −6.28 | 0.04719 |
| RASGRP4 | −6.70 | 0.00886 |
| CCL18 | −7.07 | 0.02313 |
| CRYAA | −9.32 | 0.02600 |
| C1QB | −9.80 | 0.04701 |

^{a}Log2 fold change is the log base 2 fold change of FPKM values of the gene in cancer samples against control samples based on the standard differential analysis.
^{b}q-value is the false-discovery-rate-adjusted p-value of the test statistic.

TABLE 5
Differentially expressed genes identified in comparison of high
grade bladder cancer to control based on urinary RNA-seq.

| Gene | Log2 fold change^{a} | q-value^{b} |
|---|---|---|
| CP | 7.39 | 0.00799 |
| PLEKHS1 | 5.45 | 0.00799 |
| MYBPC1 | 5.12 | 0.00799 |
| ROBO1 | 4.60 | 0.00799 |
| RARRES1 | 4.36 | 0.03586 |
| WNT5A | 4.22 | 0.01450 |
| AKR1C2 | 4.07 | 0.00799 |
| AR | 3.88 | 0.00799 |
| IGFBP5 | 3.87 | 0.00799 |
| ENTPD5 | 3.79 | 0.00799 |
| SLC14A1 | 3.76 | 0.00799 |
| FBLN1 | 3.66 | 0.00799 |
| SYBU | 3.62 | 0.00799 |
| MYB | 3.48 | 0.00799 |
| STEAP2 | 3.33 | 0.00799 |
| EEF2K | 3.19 | 0.00799 |
| GPD1L | 3.10 | 0.00799 |
| CAPN13 | 3.08 | 0.00799 |
| SIDT1 | 3.03 | 0.00799 |
| TMEM98 | 2.98 | 0.00799 |
| CLIC6 | 2.96 | 0.00799 |
| SRGAP3 | 2.96 | 0.00799 |
| TNFSF15 | 2.93 | 0.00799 |
| SPTSSB | 2.91 | 0.01450 |
| MPPED2 | 2.90 | 0.02740 |
| ENPP5 | 2.87 | 0.00799 |
| PBX1 | 2.85 | 0.00799 |
| HMGCS2 | 2.76 | 0.02387 |
| FXYD3 | 2.73 | 0.01937 |
| PEX11A | 2.72 | 0.00799 |
| MGST1 | 2.71 | 0.00799 |
| MUC15 | 2.68 | 0.00799 |
| ALDH5A1 | 2.63 | 0.00799 |
| VIPR1 | 2.61 | 0.01937 |
| GNPTAB | 2.59 | 0.02740 |
| ATP2A3 | 2.58 | 0.03887 |
| PDK3 | 2.57 | 0.00799 |
| PPM1L | 2.57 | 0.00799 |
| MBOAT1 | 2.54 | 0.00799 |
| BMP3 | 2.53 | 0.00799 |
| TTLL7 | 2.50 | 0.04119 |
| LOC92249 | 2.50 | 0.01937 |
| FAM162A | 2.48 | 0.00799 |
| AGR2 | 2.46 | 0.02387 |
| CRABP2 | 2.34 | 0.00799 |
| ZNF704 | 2.33 | 0.01450 |
| LPAR5 | 2.31 | 0.04757 |
| HERC2P9 | 2.29 | 0.04119 |
| AUTS2 | 2.29 | 0.04447 |
| TLR3 | 2.27 | 0.03887 |
| TSPAN12 | 2.26 | 0.00799 |
| RGMB | 2.22 | 0.02740 |
| SDR42E1 | 2.21 | 0.01450 |
| CDC42BPA | 2.19 | 0.03214 |
| PTGFRN | 2.19 | 0.00799 |
| FUT8 | 2.19 | 0.04119 |
| EPAS1 | 2.19 | 0.00799 |
| TLE1 | 2.13 | 0.04119 |
| WDR52 | 2.04 | 0.00799 |
| XBP1 | 2.02 | 0.00799 |
| HSD17B11 | 2.02 | 0.03887 |
| RHOU | 1.84 | 0.02387 |
| LONP2 | 1.77 | 0.00799 |
| SPICE1 | 1.74 | 0.00799 |
| LPCAT3 | 1.72 | 0.02740 |
| KIAA1244 | 1.67 | 0.02387 |
| KDELR2 | 1.66 | 0.01450 |
| HADH | 1.65 | 0.02387 |
| TTC21B | 1.62 | 0.03586 |
| PTCD3 | 1.60 | 0.01937 |
| MRFAP1L1 | 1.58 | 0.02740 |
| TTC37 | 1.52 | 0.03214 |
| EEA1 | 1.51 | 0.04119 |
| RAB3GAP1 | 1.48 | 0.02740 |
| MIDN | −2.13 | 0.03887 |
| THBS1 | −2.20 | 0.02387 |
| HIST1H2AC | −2.20 | 0.00799 |
| CREM | −2.21 | 0.00799 |
| LRG1 | −2.21 | 0.00799 |
| HCAR2 | −2.26 | 0.01937 |
| GNA15 | −2.48 | 0.00799 |
| PLEKHH2 | −2.49 | 0.01937 |
| GK | −2.49 | 0.00799 |
| CSRNP1 | −2.50 | 0.00799 |
| GADD45B | −2.54 | 0.00799 |
| SIRPA | −2.60 | 0.00799 |
| KDM6B | −2.68 | 0.00799 |
| DENND3 | −2.73 | 0.00799 |
| KCTD11 | −2.77 | 0.00799 |
| LAMC2 | −3.04 | 0.04447 |
| ICAM1 | −3.20 | 0.00799 |
| SERPINA1 | −3.26 | 0.01450 |
| CHST15 | −3.45 | 0.00799 |
| NR4A1 | −3.63 | 0.00799 |
| NREP | −3.74 | 0.01937 |
| SPP1 | −4.20 | 0.03586 |
| IFI30 | −4.96 | 0.00799 |
| MT2A | −5.00 | 0.00799 |
| PTPRC | −5.32 | 0.03586 |
| ZEB2 | −5.71 | 0.00799 |
| CD14 | −5.93 | 0.02740 |
| TYROBP | −6.14 | 0.04757 |
| RASGRP4 | −6.52 | 0.00799 |
| CCL18 | −6.77 | 0.04757 |
| CRYAA | −8.60 | 0.04447 |

^{a}Log2 fold change is the log base 2 fold change of FPKM values of the gene in cancer samples against control samples based on the standard differential analysis.
^{b}q-value is the false-discovery-rate-adjusted p-value of the test statistic.

TABLE 6
Differentially expressed genes identified in comparison of low
grade bladder cancer to control based on urinary RNA-seq.

| Gene | Log2 fold change^{a} | q-value^{b} |
|---|---|---|
| SRD5A2 | 4.91 | 0.02163 |
| IGFBP5 | 4.58 | 0.02163 |
| CFTR | 4.50 | 0.02163 |
| RARRES1 | 4.47 | 0.02163 |
| MUC13 | 4.13 | 0.03785 |
| MBOAT1 | 3.76 | 0.02163 |
| TMEM98 | 3.72 | 0.02163 |
| C1orf21 | 2.74 | 0.02163 |
| PFKFB3 | −3.09 | 0.02163 |
| BAG3 | −3.18 | 0.03785 |
| CLIC3 | −3.19 | 0.02163 |
| PLEKHG2 | −3.69 | 0.02163 |
| PTGS2 | −3.92 | 0.02163 |
| HMOX1 | −4.55 | 0.02163 |
| THBS1 | −4.58 | 0.03785 |
| PRSS22 | −4.59 | 0.02163 |
| DPP4 | −5.49 | 0.02163 |

^{a}Log2 fold change is the log base 2 fold change of FPKM values of the gene in cancer samples against control samples based on the standard differential analysis.
^{b}q-value is the false-discovery-rate-adjusted p-value of the test statistic.

TABLE 7
Differentially expressed genes identified in comparison of high
grade to low grade bladder cancer based on urinary RNA-seq.

| Gene | Log2 fold change^{a} | q-value^{b} |
|---|---|---|
| MTRNR2L8 | 8.92 | 0.04817 |
| VEGFA | 3.56 | 0.04817 |
| AKAP12 | 3.11 | 0.04817 |

^{a}Log2 fold change is the log base 2 fold change of FPKM values of the gene in HG samples against LG samples based on the standard differential analysis.
^{b}q-value is the false-discovery-rate-adjusted p-value of the test statistic.

TABLE 8
Cancer genes analyzed for construction of the diagnostic model.

| | Log2 fold | | Taqman assay |
|---|---|---|---|
| Gene Symbol | change^{a} | q-value^{b} | number^{c} |
| CP | 7.39 | 0.00799 | Hs00236810_m1 |
| BPIFB1 | 6.48 | 0.0190 | Hs00264197_m1 |
| PLEKHS1 | 5.45 | 0.00799 | Hs00913117_m1 |
| MYBPC1 | 5.12 | 0.00799 | Hs00159451_m1 |
| ROBO1 | 4.60 | 0.00799 | Hs00268049_m1 |
| RARRES1 | 4.36 | 0.0359 | Hs00161204_m1 |
| WNT5A | 4.22 | 0.0145 | Hs00998537_m1 |
| AKR1C2 | 4.07 | 0.00799 | Hs00912742_m1 |
| AR | 3.88 | 0.00799 | Hs00171172_m1 |
| IGFBP5 | 3.87 | 0.00799 | Hs00181213_m1 |
| ENTPD5 | 3.79 | 0.00799 | Hs00969100_m1 |
| SLC14A1 | 3.76 | 0.00799 | Hs00998197_m1 |
| FBLN1 | 3.66 | 0.00799 | Hs00972609_m1 |
| SYBU | 3.62 | 0.00799 | Hs01052028_m1 |
| STEAP2 | 3.33 | 0.00799 | Hs00401292_m1 |
| GPD1L | 3.10 | 0.00799 | Hs00380518_m1 |

^{a}Log2 fold change is the log base 2 fold change of FPKM values of the gene in cancer samples against control samples based on the standard differential analysis.
^{b}q-value is the false-discovery-rate-adjusted p-value of the test statistic.
^{c}Taqman assay number is the catalogue number of the Taqman gene expression assay for qPCR experiment.

TABLE 9
Reference genes analyzed for construction of the diagnostic model.

| | Average FPKM | SD of FPKM | |
|---|---|---|---|
| | values of all | values of all | Taqman assay |
| Gene Symbol | RNA-seq samples | RNA-seq samples | number^{a} |
| QRICH1 | 4.13 | 0.49 | Hs00214646_m1 |
| CDC42BPB | 4.03 | 0.39 | Hs00178787_m1 |
| USP39 | 3.89 | 0.45 | Hs01046897_m1 |
| ITSN1 | 3.87 | 0.50 | Hs00161676_m1 |
| DNMBP | 3.79 | 0.50 | Hs00324375_m1 |

^{a}Taqman assay number is the catalogue number of the Taqman gene expression assay for qPCR experiment.

TABLE 10
Univariate logistic analysis of cancer and reference
genes for development of the diagnostic model.

| | Gene | Odds Ratio (95% CI) | AUC | p-value |
|---|---|---|---|---|
| Cancer | WNT5A | 2.12 (1.63-2.96) | 0.90 | <0.0001 |
| | RARRES1 | 1.79 (1.47-2.30) | 0.90 | <0.0001 |
| | ROBO1 | 1.70 (1.42-2.14) | 0.92 | <0.0001 |
| | CP | 1.65 (1.39-2.06) | 0.94 | <0.0001 |
| | IGFBP5 | 1.43 (1.26-1.69) | 0.89 | <0.0001 |
| | PLEKHS1 | 1.37 (1.24-1.57) | 0.92 | <0.0001 |
| | BPIFB1 | 1.32 (1.20-1.49) | 0.89 | <0.0001 |
| | MYBPC1 | 1.29 (1.18-1.45) | 0.87 | <0.0001 |
| Reference | DNMBP | 1.58 (1.29-2.02) | 0.74 | <0.0001 |
| | QRICH1 | 1.18 (0.97-1.45) | 0.65 | 0.0923 |
| | CDC42BPB | 1.14 (1.00-1.33) | 0.61 | 0.0476 |

While the preferred embodiments of the invention have been illustrated and described, it will be appreciated that various changes can be made therein without departing from the spirit and scope of the invention.

What is claimed is:

1. A method for diagnosing and treating bladder cancer in a subject, the method comprising:
a) collecting a urine sample from the subject;
b) isolating urinary cells from the urine sample;
c) measuring levels of expression of ROBO1 and WNT5A biomarkers in the urinary cells;
d) diagnosing the subject by analyzing the levels of expression of each biomarker in conjunction with respective reference value ranges for the biomarkers, wherein increased levels of expression of the ROBO1 and WNT5A biomarkers compared to the reference value ranges for the biomarkers for a control subject indicate that the subject has bladder cancer; and
e) administering an anti-cancer treatment for the bladder cancer to the subject if the subject is diagnosed with bladder cancer, wherein the anti-cancer treatment comprises surgical removal of the bladder cancer, immunotherapy, or chemotherapy.

2. The method of claim 1, further comprising removing white blood cells and red blood cells from the urine sample prior to isolating the urinary cells.

3. The method of claim 1, further comprising measuring a level of expression of at least one reference marker selected from the group consisting of QRICH1, CDC42BPB and DNMBP, wherein the level of expression of the at least one reference marker is used for data normalization.

4. The method of claim 1, wherein the immunotherapy comprises administration of an effective amount of Bacillus Calmette-Guerin (BCG).

5. The method of claim 1, wherein the surgical removal of the bladder cancer comprises transurethral resection or cystectomy.

6. The method of claim 1, wherein the chemotherapy comprises administration of a therapeutically effective amount of mitomycin, valrubicin, docetaxel, thiotepa, or gemcitabine.

7. The method of claim 6, wherein the chemotherapy comprises intravesical therapy or electromotive therapy.

8. The method of claim 1, further comprising measuring levels of expression of one or more biomarkers selected from the group consisting of RARRES1, CP, IGFBP5, PLEKHS1, BPIFB1, and MYBPC1, wherein increased levels of expression of the ROBO1 and WNT5A biomarkers in combination with increased levels of expression of the one or more biomarkers selected from the group consisting of RARRES1, CP, IGFBP5, PLEKHS1, BPIFB1, and MYBPC1 compared to reference value ranges for the biomarkers for a control subject indicate that the subject has bladder cancer.

9. The method of claim 1, further comprising measuring levels of expression of RARRES1 and CP, wherein increased levels of expression of the ROBO1 and WNT5A biomarkers in combination with increased levels of expression of the RARRES1 and CP biomarkers compared to reference value ranges for the biomarkers for a control subject indicate that the subject has bladder cancer.

10. The method of claim 1, further comprising measuring levels of expression of one or more additional genes selected from Tables 4-10 in the urinary cells, wherein increased levels of expression of the ROBO1 and WNT5A in combination with differential expression of the one or more additional genes selected from Tables 4-10 compared to reference value ranges for the levels of expression of the genes for the control subject indicate that the subject has bladder cancer.

11. The method of claim 1, further comprising measuring levels of expression of one or more additional genes selected from Tables 5 and 6 in the urinary cells, and distinguishing whether the subject has low-grade bladder cancer or high-grade bladder cancer by comparing the levels of expression of the one or more genes selected from Tables 5 and 6 to reference value ranges for subjects having low-grade bladder cancer or high-grade bladder cancer.

12. The method of claim 11, comprising measuring levels of expression in the urinary cells of one or more genes selected from Table 5, wherein differential expression of the one or more genes selected from Tables 5 compared to reference value ranges for a control subject indicate that the subject has high grade bladder cancer.

13. The method of claim 11, comprising measuring levels of expression in the urinary cells of one or more genes selected from Table 6, wherein differential expression of the one or more genes selected from Tables 6 compared to reference value ranges for a control subject indicate that the subject has low grade bladder cancer.

14. The method of claim 11, comprising measuring levels of expression of one or more genes selected from the group consisting of MTRNR2L8, VEGFA, and AKAP12 in the urinary cells, wherein increased levels of expression of the one or more genes selected from the group consisting of MTRNR2L8, VEGFA, and AKAP12 compared to reference value ranges for a subject having low grade bladder cancer indicates that the subject has high grade bladder cancer and decreased levels of expression of the one or more genes selected from the group consisting of MTRNR2L8, VEGFA, and AKAP12 compared to reference value ranges for a subject having high grade bladder cancer indicates that the subject has low grade bladder cancer.

15. A method of performing endoscopy screening for bladder cancer, the method comprising:
a) collecting a urine sample from the subject;
b) isolating urinary cells from the urine sample;
c) measuring levels of expression of ROBO1 and WNT5A biomarkers in the urinary cells;
d) analyzing the levels of expression of each biomarker in conjunction with respective reference value ranges for the biomarkers, wherein increased levels of expression of the ROBO1 and WNT5A biomarkers compared to the reference value ranges for the biomarkers for a control subject indicate that the subject has bladder cancer; and
e) performing the endoscopy screening on the subject if the levels of expression of the ROBO1 and WNT5A biomarkers indicate that the subject has bladder cancer, or reducing the frequency of the endoscopy screening for bladder cancer if the levels of expression of the ROBO1 and WNT5A biomarkers indicate that the subject does not have bladder cancer.

16. The method of claim 15, wherein reducing the frequency of the endoscopy screening comprises waiting to perform endoscopy screening until the levels of expression of the ROBO1 and WNT5A biomarkers compared to the reference value ranges for the biomarkers indicate that the subject has bladder cancer.

17. The method of claim 15, wherein reducing the frequency of endoscopy screening comprises performing endoscopy screening once a year, every other year, or every 2, 3, 4, or 5 years if the levels of expression of the ROBO1 and WNT5A biomarkers compared to the reference value ranges for the biomarkers indicate that the subject does not have bladder cancer.

18. The method of claim 15, wherein the subject is at risk of having bladder cancer because of smoking, chronic catheterization, or an environmental exposure to a carcinogen.

19. The method of claim 15, wherein the subject is a veteran, firefighter, chemist, bus driver, rubber worker, mechanic, leather worker, blacksmith, machine setter, or hairdresser.

20. The method of claim 15, further comprising removing white blood cells and red blood cells from the urine sample prior to isolating the urinary cells.

21. The method of claim 15, further comprising measuring a level of expression of at least one reference marker selected from the group consisting of QRICH1, CDC42BPB and DNMBP, wherein the level of expression of the at least one reference marker is used for data normalization.

22. The method of claim 15, further comprising measuring levels of expression of one or more biomarkers selected from the group consisting of RARRES1, CP, IGFBP5, PLEKHS1, BPIFB1, and MYBPC1, wherein increased levels of expression of the ROBO1 and WNT5A biomarkers in combination with increased levels of expression of the one or more biomarkers selected from the group consisting of RARRES1, CP, IGFBP5, PLEKHS1, BPIFB1, and MYBPC1 compared to reference value ranges for the biomarkers for a control subject indicate that the subject has bladder cancer; and performing the endoscopy screening on the subject if the levels of expression of the ROBO1 and WNT5A biomarkers in combination with the levels of expression of the one or more biomarkers selected from the group consisting of RARRES1, CP, IGFBP5, PLEKHS1, BPIFB1, and MYBPC1 indicate that the subject has bladder cancer, or reducing the frequency of the endoscopy screening for bladder cancer if the levels of expression of the ROBO1 and WNT5A biomarkers in combination with the levels of expression of the one or more biomarkers selected from the group consisting of RARRES1, CP, IGFBP5, PLEKHS1, BPIFB1, and MYBPC1 biomarkers indicate that the subject does not have bladder cancer.

23. The method of claim 15, further comprising measuring levels of expression of RARRES1 and CP biomarkers, wherein increased levels of expression of the ROBO1 and WNT5A biomarkers in combination with increased levels of expression of the RARRES1 and CP biomarkers compared to reference value ranges for the biomarkers for a control subject indicate that the subject has bladder cancer; and performing the endoscopy screening on the subject if the levels of expression of the ROBO1, WNT5A, RARRES1 and CP biomarkers indicate that the subject has bladder cancer, or reducing the frequency of the endoscopy screening for bladder cancer if the levels of expression of the ROBO1, WNT5A, RARRES1 and CP biomarkers indicate that the subject does not have bladder cancer.

24. A method for monitoring the efficacy of a therapy for treating bladder cancer in a subject, the method comprising: measuring levels of expression of MTRNR2L8, VEGFA, and AKAP12 biomarkers in a first sample derived from the subject before the subject undergoes said therapy and a second sample derived from the subject after the subject undergoes said therapy, wherein increased levels of expression of the MTRNR2L8, VEGFA, and AKAP12 biomarkers in the second sample compared to the levels of expression of the biomarkers in the first sample indicate that the subject is worsening, and decreased levels of expression of the MTRNR2L8, VEGFA, and AKAP12 biomarkers in the second sample compared to the levels of expression of the biomarkers in the first sample indicate that the subject is improving.

25. The method of claim 24, further comprising measuring a level of expression of at least one reference marker selected from the group consisting of QRICH1, CDC42BPB and DNMBP, wherein the level of expression of the at least one reference marker is used for data normalization.

26. The method of claim 24, further comprising measuring levels of expression of one or more biomarkers selected from the group consisting of ROBO1, WNT5A, RARRES1, CP, IGFBP5, PLEKHS1, BPIFB1, and MYBPC1 in the first sample derived from the subject before the subject undergoes said therapy and the second sample derived from the subject after the subject undergoes said therapy, wherein increased levels of expression of the MTRNR2L8, VEGFA, and AKAP12 biomarkers in combination with increased levels of expression of the one or more biomarkers selected from the group consisting of ROBO1, WNT5A, RARRES1, CP, IGFBP5, PLEKHS1, BPIFB1, and MYBPC1 in the second sample compared to the levels of expression of the biomarkers in the first sample indicate that the subject is worsening, and decreased levels of expression of the MTRNR2L8, VEGFA, and AKAP12 biomarkers in combination with decreased levels of expression of the one or more biomarkers selected from the group consisting of ROBO1, WNT5A, RARRES1, CP, IGFBP5, PLEKHS1, BPIFB1, and MYBPC1 in the second sample compared to the levels of expression of the biomarkers in the first sample indicate that the subject is improving.

27. The method of claim 24, further comprising measuring levels of expression of RARRES1 and CP biomarkers in the first sample derived from the subject before the subject undergoes said therapy and the second sample derived from the subject after the subject undergoes said therapy, wherein increased levels of expression of the ROBO1 and WNT5A biomarkers in combination with increased levels of expression of the RARRES1 and CP biomarkers in the second sample compared to the levels of expression of the biomarkers in the first sample indicate that the subject is worsening, and decreased levels of expression of the ROBO1 and WNT5A biomarkers in combination with decreased levels of expression of the RARRES1 and CP biomarkers in the second sample compared to the levels of expression of the biomarkers in the first sample indicate that the subject is improving.

28. A method of distinguishing whether a subject has low-grade bladder cancer or high-grade bladder cancer and treating the subject for bladder cancer, the method comprising:
a) collecting a urine sample from the subject;
b) isolating urinary cells from the urine sample;
c) measuring levels of expression of the one or more genes selected from the group consisting of MTRNR2L8, VEGFA, and AKAP12 in the urinary cells;
d) distinguishing whether the subject has low-grade bladder cancer or high-grade bladder cancer by analyzing the levels of expression of the one or more genes selected from the group consisting of MTRNR2L8, VEGFA, and AKAP12 in conjunction with respective reference value ranges for subjects with low-grade bladder cancer or high-grade bladder cancer, wherein increased levels of expression of the one or more genes selected from the group consisting of MTRNR2L8, VEGFA, and AKAP12 compared to the reference value ranges for a subject having low grade bladder cancer indicate that the subject has high grade bladder cancer and decreased levels of expression of the one or more genes selected from the group consisting of MTRNR2L8, VEGFA, and AKAP12 compared to the reference value ranges for a subject having high grade bladder cancer indicate that the subject has low grade bladder cancer; and
e) administering an anti-cancer treatment for high grade bladder cancer to the subject if the subject is diagnosed with high grade bladder cancer, and administering an anti-cancer treatment for low grade bladder cancer to the subject if the subject is diagnosed with low grade bladder cancer.

29. The method of claim 28, further comprising measuring levels of expression of one or more additional genes selected from Tables 5 and 6 in the urinary cells, and comparing the levels of expression of the one or more additional genes selected from Tables 5 and 6 to reference value ranges for subjects having low-grade bladder cancer or high-grade bladder cancer.
